SEQUENCE LISTING (12 sequences)

SEQ ID NO: 1 - DNA, 20 bp, Homo sapiens
gagacgtaatccagcattgg

SEQ ID NO: 2 - DNA, 21 bp, Homo sapiens
ctgaggtctgctatttactgg

SEQ ID NO: 3 - DNA, 21 bp, Homo sapiens
ccagtaaatagcagacctcag

SEQ ID NO: 4 - DNA, 21 bp, Homo sapiens
gactagattgactcagtgcac

SEQ ID NO: 5 - DNA, 21 bp, Homo sapiens
gtgcactgagtcaatctagtc

SEQ ID NO: 6 - DNA, 20 bp, Homo sapiens
taactttctccactgggatg

SEQ ID NO: 7 - DNA, 27 bp, Homo sapiens
actcgaggagacgtaatccagcattgg

SEQ ID NO: 8 - DNA, 29 bp, Homo sapiens
agcggccgctaactttctccactgggatg

SEQ ID NO: 9 - DNA, 31 bp, Homo sapiens
catatgaatgaacgttcgtagcagttgcttc

SEQ ID NO: 10 - DNA, 31 bp, Homo sapiens
gaagcaactgctactgcctttcattcatatg

SEQ ID NO: 11 - RNA, 22 nt, Homo sapiens
uggcagugucuuagcugguugu

SEQ ID NO: 12 - RNA, 21 nt, Homo sapiens
cuuuuugcggucugggcuugc

(19) United States
(12) Patent Application Publication
(10) Pub. No.: US 2021/0155928 A1
(43) Pub. Date: May 27, 2021

(54) RNA-AIDED IMMUNOTHERAPEUTICS

(71) Applicants: Beth Israel Deaconess Medical Center, Boston, MA (US); La Sapienza University, Rome (IT)

(72) Inventors: Frank SLACK, Boston, MA (US); Eleni ANASTASIADOU, Boston, MA (US); Pankaj TRIVEDI, Rome (IT)

(21) Appl. No.: 17/059,802

(22) PCT Filed: May 30, 2019
(86) PCT No.: PCT/US2019/034573

§ 371 Date: Nov. 30, 2020

Related U.S. Application Data
(60) Provisional application No. 62/678,728, filed on May 31, 2018.

(51) Int. Cl. C12N 15/113; A61P 35/00; A61K 31/7105; A61K 9/00; A61K 39/395

(52) U.S. Cl. CPC C12N 15/113; A61P 35/00; A61K 31/7105; A61K 9/0019; A61K 39/3955; C12N 2320/35; C12N 2320/32; C12N 2320/31; C12N 2310/141; C12N 2310/113; C12N 2310/11

(57) ABSTRACT

The present disclosure provides materials and methods for treating cancer, including Epstein-Barr virus-related cancer.

### CROSS-REFERENCE TO RELATED APPLICATIONS

This application claims priority to U.S. Provisional Application No. 62/678,728, filed on May 31, 2018, the entire contents of which are incorporated herein.

### DESCRIPTION OF THE TEXT FILE SUBMITTED ELECTRONICALLY

The instant application contains a Sequence Listing which has been submitted in ASCII format via EFS-Web and is hereby incorporated by reference in its entirety. Said ASCII copy, created about May 21, 2019, is named “BID-007PC_ST25.txt” and is about 2.17 KB in size.

### FIELD OF THE DISCLOSURE

The disclosure is directed to materials and methods for treating cancer with RNA-based agents.

### BACKGROUND

Cancer is a significant health problem worldwide. In spite of recent advances that have been made in detection and therapy of cancer there is no universally successful method for prevention or treatment presently available. Existing therapies, which are generally based on a combination of chemotherapy, surgery and radiation are insufficient for many patients.

In spite of considerable research into therapies for Epstein-Barr virus (EBV) related cancer and other cancers, EBV-associated lymphomas remain difficult to diagnose and treat effectively. Among Non-Hodgkin lymphoma (NHL), more than 95% of endemic B cell lymphomas (BL) are associated with EBV. Diffuse large B-cell lymphomas (DLBCLs) constitute about 30% of all NHLs, of which about 10%, are EBV associated in immunocompetent patients. EBV's ability to cause cancer, inter alia, lies in its capacity to evade host immune surveillance by altering immune checkpoints (IC). Increased understanding of the immune tumor microenvironment has allowed for investigation and the development of new agents that target immune pathways for therapy. Similarly, expression of miRNAs, which are small noncoding RNAs which post-transcriptionally regulate gene expression is altered in a broad range of cancers, including EBV-related cancers. Therefore, there is a critical need to develop more effective therapies and treatment methods for cancers, including EBV-related cancers, that alter ICs and regulate cellular miRNAs. The present disclosure fulfills these needs and further provides other related advantages.

### SUMMARY

Immunotherapy of cancers is a desirable therapeutic alternative in lieu of or in addition to the standard chemotherapy and radiation therapy protocols. Immune checkpoint inhibitors have been satisfactory in overall response rate for several different tumors and in particular EBV-associated tumors. In the context of EBV-associated tumors, viral proteins like EBNA2 and LMP1 affect immune checkpoint genes, such as, PD-L1 and ICOSL expression by altering miRNAs. Thus, a combination of miRNA, their mimics or chemically modified antisense oligonucleotides targeting miRNAs (i.e., a locked nucleic acid (LNA)), and immune checkpoints inhibitors on nanoparticles, provide a unique method for silencing immune checkpoints both from outside and within the tumor cell.

In one aspect, the present disclosure provides a method for treating an EBV-related cancer in a subject in need thereof, comprising administering an effective amount of one or more of (a) an agent that increases an amount of miR-34a in the subject and (b) an agent that decreases an amount of miR-129 in the subject, wherein: the subject is undergoing treatment with an immune checkpoint immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

In some embodiments, the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), and adult T-cell leukemia (ATL)/lymphoma.

In some embodiments, the agent that increases an amount of miR-34a is selected from one or more of miR-34a and a miR-34a mimetic. In some embodiments, the agent that increases an amount of miR-34a is an inhibitor of Early B-cell factor (EBF1).

In some embodiments, the agent that decreases an amount of miR-129 is selected from one or more of an antisense oligonucleotide, an antagomir and a construct expressing a miRNA inhibitor. In some embodiments, the antisense oligonucleotide comprises a sequence that is at least partially complementary to a mature sequence of miR-129. In some embodiments, the agent is chemically modified. In some embodiments, the chemical modification is selected from locked nucleic acid (LNA), phosphorothioate, 2′-O-Methyl, 2′-O-Methoxyethyl, 2′-O-alkyl-RNA unit, 2′-OMe-RNA unit, 2′-amino-DNA unit, 2′-fluoro-DNA unit, peptide nucleic acid (PNA) unit, hexitol nucleic acids (HNA) unit, INA unit, and a 2′-O-(2-Methoxyethyl)-RNA (2′ MOE RNA) unit.

In some embodiments, the agent that modulates PD-1 is an antibody or antibody format specific for PD-1. In some embodiments, the antibody or antibody format specific for PD-1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In some embodiments, the antibody or antibody format specific for PD-1 is selected from Nivolumab, Pembrolizumab, Pidilizumab, BMS-936559, Atezolizumab, or Avelumab.

In some embodiments, the agent that modulates PD-L1 is an antibody or antibody format specific for PD-L1. In some embodiments, the antibody or antibody format specific for PD-L1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In some embodiments, the antibody or antibody format specific for PD-L1 is selected from Nivolumab, Pembrolizumab, Pidilizumab, BMS-936559, Atezolizumab, Avelumab or Durvalumab.

In some embodiments, the agent that modulates PD-L2 is an antibody or antibody format specific for PD-L2. In some embodiments, the antibody or antibody format specific for PD-L2 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

In some embodiments, the agent that modulates ICOS is an antibody or antibody format specific for ICOS. In some embodiments, the antibody or antibody format specific for ICOS is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In some embodiments, the antibody or antibody format specific for ICOS comprises JTX-2011.

In some embodiments, the agent that modulates ICOSL is an antibody or antibody format specific for ICOSL. In some embodiments, the antibody or antibody format specific for ICOSL is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

In some embodiments, the agent that modulates CTLA-4 is an antibody or antibody format specific for CTLA-4. In some embodiments, the antibody or antibody format specific for CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody. In some embodiments, the antibody or antibody format specific for CTLA-4 is selected from tremelimumab or Ipilimumab.

In some embodiments, administration is by intratumoral, intradermal, subcutaneous, intramuscular, intraperitoneal or intravenous injection, or direct injection into cancer tissue.

In one aspect, the disclosure provides a method for treating an EBV-related cancer in a subject in need thereof, comprising administering (i) an effective amount of one or more of (a) an agent that increases an amount of miR-34a in the subject and (b) an agent that decreases an amount of miR-129 in the subject, and (ii) an effective amount of an immune checkpoint immunotherapy selected from an agent that modulates one or more of PD-1, PD-L1, PD-L2, ICOS, ICOSL, and CTLA-4.

In one aspect, the disclosure provides a method for potentiating an immune checkpoint immunotherapy of an EBV-related cancer in a subject in need thereof, comprising administering an agent that increases an amount of miR-34a in the subject, wherein: the immune checkpoint immunotherapy is an agent that modulates one or more of PD-1, PD-L1, and PD-L2 and the subject is predicted to be poorly responsive or non-responsive to the immune checkpoint immunotherapy or has presented as poorly responsive or non-responsive to the immune checkpoint immunotherapy.

In one aspect, the disclosure provides a method for potentiating immune checkpoint immunotherapy of an EBV-related cancer in a subject in need thereof, comprising administering an agent that decreases an amount of miR-129 in the subject, wherein the immune checkpoint immunotherapy is an agent that modulates one or more of ICOS, ICOSL, and CTLA-4 and the subject is predicted to be poorly responsive or non-responsive to the immune checkpoint immunotherapy or has presented as poorly responsive or non-responsive to the immune checkpoint immunotherapy.

In some embodiments, the method reduces and/or mitigates one or more side effects of the immune checkpoint immunotherapy. In some embodiments, the side effect is selected from decreased appetite, rashes, fatigue, pneumonia, pleural effusion, pneumonitis, pyrexia, nausea, dyspnea, cough, constipation, diarrhea, immune-mediated pneumonitis, colitis, hepatitis, endocrinopathies, hypophysitis, iridocyclitis, and nephritis.

In some embodiments, the method reduces the dose of immune checkpoint immunotherapy. In some embodiments, method reduces number of administrations of the immune checkpoint immunotherapy. In some embodiments, the method increases a therapeutic window of the immune checkpoint immunotherapy.

In some embodiments, the method elicits a potent immune response in less-immunogenic tumors. In some embodiments, the method converts a tumor with reduced inflammation (“cold tumor”) to a responsive, inflamed tumor (“hot tumor”).

In some embodiments, the method makes the cancer responsive or more responsive to a combination therapy of the immune checkpoint immunotherapy and one or more chemotherapeutic agents and/or radiotherapy. In some embodiments, the subject is predicted to be poorly responsive or non-responsive to the immune checkpoint immunotherapy based on expression of one or more of PD-1, PD-L1, PD-L2, ICOS, ICOSL, and CTLA-4 in a tumor specimen. In some embodiments, the subject is predicted to be poorly responsive or non-responsive to an agent that modulates one or more of PD-1, PD-L1, and PD-L2 based on low on expression of PD-1, PD-L1, and PD-L2 in a tumor specimen.

In some embodiments, the subject is predicted to be poorly responsive or non-responsive to an agent that modulates one or more of PD-1, PD-L1, and PD-L2 tumor proportion score (TPS) of less than about 49% for PD-L1 staining.

In one aspect, the disclosure provides a method for treating an EBV-related cancer in a subject in need thereof, comprising administering (i) an effective amount of one or more of (a) an agent that increases an amount of miR-34a in the subject and (b) an agent that decreases an amount of miR-129 in the subject, and (ii) an effective amount of an immune checkpoint immunotherapy selected from an agent that modulates one or more of PD-1, PD-L1, PD-L2, ICOS, ICOSL, and CTLA-4.

In one aspect, the disclosure provides a method for evaluating an EBV-related cancer subject's likelihood of response to an immune checkpoint immunotherapy, comprising evaluating a level of one or more of miR-34a and miR-129 in a biological sample from the subject, wherein a low level of miR-34a and/or high level of miR-129 is indicative of a cancer that is suitable for immune checkpoint immunotherapy.

In one aspect, the disclosure provides a method for treating an EBV-related cancer, comprising: (a) evaluating a subject's likelihood of response to an immune checkpoint immunotherapy, comprising evaluating a level of one or more of miR-34a and miR-129 in a biological sample from the subject, wherein a low level of miR-34a and/or high level of miR-129 is indicative of a cancer that is suitable for immune checkpoint immunotherapy and (b) administering an immune checkpoint immunotherapy selected from an agent that modulates one or more of PD-1, PD-L1, and PD-L2 based on low on expression of PD-1, PD-L1, and PD-L2 to the subject having a low level of miR-34a and/or high level of miR-129.

### BRIEF DESCRIPTION OF THE DRAWINGS

FIG. 1A-E are a series of western blot images showing PD-L1 expression in EBV infected and EBNA2 transfected BL and DLBCL cell lines. FIG. 1A: EBNA2 and PD-L1 expression in Mutu I (latency I expressor) and its latency III expressing counterpart. Furthermore, expression of PD-L1 and EBNA2 is shown in two additional BLs with resident viral genomes. Daudi cells carry an EBNA2 deleted EBV strain, and Jijoye cells are EBNA2 positive BL (see gel on the right of FIG. 1A). FIG. 1B shows two GC DLBCLs, namely U2932 and SUDHL5 cells were infected with a recombinant Akata strain of EBV. Total cell lysates were electrophoresed and EBNA2 expression was verified by immunoblotting using PE2 monoclonal antibodies. PD-L1 expression was analyzed using rat monoclonal antibodies. FIG. 1C shows two BL cell lines, Oma4 and DG75, which were infected with the recombinant Akata virus and tested for EBNA2 and PD-L1 expression. (3-actin was used as loading control. FIG. 1D shows PD-L1, EBNA2 and LMP1 expression in transfected U2932 DLBCL and FIG. 1E shows BL41 is an EBV negative BL. The BL41K3 derivatives are estrogen inducible EBNA2 transfectants. PD-L1, EBNA2 and f3-actin expression was analyzed before and after f3-estradiol treatment. (3-actin was used as loading control.

FIG. 2A-B are a series of bar graphs showing that EBNA2 expression decreases miR-34a in transfected B lymphoma cells by affecting its transcription. miR-34a (top panels) and pre-miR-34a expression (middle panels) was verified by real time qPCR in FIG. 2A: U2932 DLBCL were transfected with EBNA2 and FIG. 2B: BL41K3 were transfected with estrogen inducible EBNA2. miR-34a promoter carrying Luc reporter activity was analyzed in U2932 and BL41 and their EBNA2 expressing derivatives (lower panels). The figure shows standard deviation (SD) of the average of three different experiments. Each experiment was performed in biological triplicates for each sample and repeated three times. P values were calculated with unpaired t test. In all cell lines miR-34a expression (****) p<0.0001. Pre-miR-34a: U2932 EBNA2 CL-1 (**) p=0.0020 and cl-2 (**) p=0.0027, BL41K3 (****) p<0.0001. MiR-34a promoter: U2932 EBNA2 CL-1 (***) p=0.0002 and cl-2 (**) p=0.0011, BL41K3 (****) p<0.0001.

FIG. 3A-B are a pair of bar graphs showing that miR-34a targets the 3′UTR of PD-L1 in EBNA2 transfected U2932 cells, and that site-directed mutagenesis of its seed sequence abrogates its binding to the PD-L1 3′ UTR. FIG. 3A is a Luciferase reporter construct containing wild type 3′ UTR PD-L1, which was transfected in the presence of either an miR-34a inhibitor in MPA vector control transfectants, or miR-34a mimic in U2932 EBNA2 expressing clone. Each transfection was performed in triplicate. For U2932 EBNA2 CL-1, (*) p=0.0172. FIG. 3B shows the specificity of miR-34a binding to its seed sequence in 3′UTR of PD-L1, which was confirmed by mutating the seed sequence by site-directed mutagenesis. The mimic miR-34a bound to the wild type 3′ UTR of PD-L1 and reduced luc activity. The inhibitory effect of mimic miR-34a was abrogated when its seed sequence in PD-L1 3′ UTR was mutated. Each transfection was performed in triplicate. (**) p=0.0026 refers to U2932 MPA Vector and (**) p=0.0021 refers to U2932 EBNA2 CL-1. P values were calculated with unpaired t test.

FIG. 4A-B show that over-expression of miR-34a in EBNA2 expressing U2932 downregulates PD-L1. FIG. 4A shows miR-34a mimic was transfected into U2932 MPA vector and EBNA2 expressing clone 1. The transfected cells were processed for PD-L1 expression by flow cytometry. FIG. 4A lower panel indicates an average of three flow cytometry experiments and its significance. (*) p=0.0109. FIG. 4B shows the effect of miR-34a reconstitution on PD-L1 expression in the vector control, and EBNA2 transfected U2932 cells was also confirmed by immunoblotting. An average of the corresponding densitometric analysis on three such experiments is shown in the lower panel, (**) p=0.0055. (3-actin served as loading control.

FIG. 5A-C show that EBNA2 suppresses miR-34a transcription through EBF1. FIG. 5A shows prediction of EBF1 binding motifs at the miR-34a promoter in the human reference genome hg38, coordinates chr1:9181678-9182943, was performed with the JASPAR database, and visualized with IGV. EBNA2 peak overlaps with the second of the predicted EBF1 binding sites (see black arrow in FIG. 5A pointing to boxed square), on the miR-34a promoter in dataset GSM2039170. FIG. 5B shows the knock-down of EBF1 was verified by Q-PCR in the U2932 cell line and U2932 EBNA2 CL-1 transduced with pLK0.1 lentiviral vectors which carry shEBF1 and control shRNA. Upon EBF1 depletion (***p=0.0004) in U2932 EBNA2 CL-1, expression of mature miR-34a (**p=0.0023) and pre-miR-34a (**p=0.0024) was depressed and consequently PD-L1 expression decreased (***p=0.0008). Q-PCRs were performed with three biological and technical triplicates for each sample. FIG. 5C shows MiR-34a promoter activity was enhanced upon EBF1 knock down in U2932 EBNA2 CL-1, after 48 h. Luciferase assay was performed three times and each sample was in triplicate. (*p=0.0105). Statistical analysis was performed using an unpaired T test (Prism-7).

FIG. 6A-C shows that miR-34a relieves suppression of immunogenicity induced by EBNA2 as measured in MLR and 3D biomimetic microfluidic coculture devices. FIG. 6A shows T cells were activated in plates coated with anti-CD3/anti-CD28 antibodies for 72 hours. The left bar in each set of bars throughout FIG. 6A shows IFN-γ in CD8 T cells, and the right bar in each set throughout FIG. 6A shows IFN-γ in CD4 T cells. Irradiated targets U2932 MPA vector and U2932 EBNA2 CL-1 were cocultivated with activated T cells (effector). The effector-target ratio was 1:10. The target cells were transfected with mimic control (right bar) or miR-34a mimic (left bar) 24 hours prior to cocultivation with the effector cells. The coculture was carried out for 48 hours and the cells were stained for CD4/CD8 and IFN-γ and processed for flow cytometry. Data are expressed as mean±SD. The P values for T cell activation without stimulators are (*) P>0.05 for CD8 and CD4. In MLR with U2932 stimulators, the statistical significance is (*) P=0.028 for CD8 and (**) P=0.0081 for CD4. Three different experiments were performed with PBMCs isolated from three different donors. FIG. 6B shows the three dimensional biomimetic microfluidic coculture devices: Four million/ml U2932 EBNA2 cells transduced with lentiviral vector controls were introduced into the microfluidic devices. In coculture experiments (right panel in FIG. 6B), devices were first seeded with U2932 EBNA2 cells and were incubated for 24 hrs at 37° C., followed by activated T cells seeding. Immunostaining was performed after 48 hours of cocultivation. In FIG. 6B, panel (i) shows representative confocal images of U2932 EBNA2 CL-1 transduced with GFP-lentiviral vector control in the absence or presence of activated T cells; in FIG. 6B, panel (ii) shows four million/ml U2932 EBNA2 CL-1, transduced with miR-34a lentivirus, were introduced in the collagen/fibronectin devices either alone (left panel (ii)-FIG. 6B) or cocultivated with previously activated T cells (right panel (ii)-FIG. 6B). The cocultivation of target, miR-34a transduced U2932 EBNA2 cells with activated T cells, was carried out for 48 hours before immunostaining. miR-34a containing lentivirus transduced U2932 EBNA2 CL-1 cells were stained with anti-GFP antibody (green-second panel), activated CD8/CD4 T cells were stained with anti-CD8 and anti-CD4 (magenta-fourth panel), apoptotic U2932 EBNA2 cl-1 cells were visualized with anti-caspase-3 antibody (red-third panel), and nuclei were counterstained with DAPI (blue-first panel). The overlap of miR-34a transduced U2932 EBNA2 cells (GFP positive) and caspase-3 (red-third panel) indicates tumor cell death (merged images, yellow-fifth panel). Scale bar=100 μm, magnification of the insets=scale bar 20 μm. FIG. 6C: Arbitrary Caspase-3 units were calculated for each experimental condition. Statistically significant caspase-3 positive cells were observed only in miR-34a transduced EBNA2 expressors; data expressed as mean±SEM; (****) p<0.0001. N=4 fields (3-4 devices for each experimental condition). Shown is one representative experiment out of 4 performed. Statistical analysis was performed with Prism-7 software using unpaired T test.

FIG. 7A-B show PD-L1 expression in DLBCL clinical tissues. FIG. 7A shows three non-GC DLBCL patient samples representing the three ABC DLBCL categories, out of a total of 21, stainings for PD-L1 are shown. Paraffin sections were immunostained for PDL1 using an Automated immunostainer (DAKO, Glostrup, Denmark). As a control for PDL-1 immunostaining, sections from paraffin embedded human lung carcinoma were used. FIG. 7B shows the stained tissue sections, which were digitalized at a 40× magnification using Aperio Scan Scope. The percentage of positivity was calculated by counting positive cells in three squared areas measuring 50000 μm2 from each clinical sample in FIG. 7A. The number of positive cells was determined using Aperio software IHC Membrane v1. This algorithm detects membrane staining for individual tumor cell in the selected regions and quantifies the intensity and completeness of the membrane staining. Unpaired t test was applied to demonstrate that differences in % total PD-L1 positive cells and % cells with strong staining intensity were statistically significant, (*) p=0.0125, (**) p=0.0040.

FIG. 8 is a western blot image showing that expression of PD-L1 in LMP-1 transfected clones; in SUDHL5 LMP1 transfected cells, high LMP1 expression does not increase PD-L1.

FIG. 9A shows detection of PD-L1 by flow cytometry in U2932 and its EBNA2 expressing derivatives and FIG. 9B shows PD-L1, miR-34a and pre-miR-34a by real-time qPCR in hormone inducibleEBNA2 transfected ER/EB 2.5 cells. FIG. 9A is a flow cytometry showing one representative experiment out of five. MFI: Mean fluorescence intensity. FIG. 9B shows PD-L1, pre-miR-34a and miR-34a expression, which was analyzed by real time q-PCR in estradiol treated ER/EB2.5 cells. *** p=0.0002 and **** p<0.0001. q-PCR was repeated three times.

FIG. 10A-B shows expression of miR-34a in U2932 and BL41 cells compared to normal CD19+ Bcells. FIG. 10A shows that MiR-34a was highly expressed in U2932 and BL41 cell lines, while miR-34a expression was reduced in EBNA2 transfected cells, compared to CD19+ B-cells from healthy donors. mean±SD of three different experiments. FIG. 10B shows that Wild type (WT) 3′UTR PD-L1 luciferase activity increases in the presence of EBNA2 in both cell lines, U2932 and BL41, confirming the lack of endogenous miR-34a binding to the seed sequence of the 3′UTR of PD-L1. Contrarily, endogenous miR-34a in both parental cell lines reduced the luciferase activity of WT 3′UTR of PD-L1. The luciferase activity of 3′UTR of PD-L1 mutated in miR-34a seed sequence was not affected. The experiments were performed three times and in triplicates. The results are shown as the mean±SD.

FIG. 11 is a bar graph showing the detection of miR-34a activity in U2932 MPA vector and U2932 EBNA2 cells cotransfected with miR-34a mimic, or mimic control. U2932 MPA vector and U2932 EBNA2 CL-1 were co-transfected with miR-34a and miR-34a mismatch biosensor in combination with mimic control or miR-34a mimic. At 48 hours (h) post-transfection, cells were analyzed for miR-34a luciferase activity. A strong reduction of luc activity was observed in both U2932 MPA vector and EBNA2 CL-1 co-transfected with mimic-miR-34a and miR-34a biosensor. This confirms both specificity and successful delivery of miR-34a in these cells, which were used for further experiments (flow cytometry, Western blot and apoptosis assay). Each sample was transfected in triplicate and the experiment was repeated at least three times. Error bars represent SEM; (***) p=0.0003 for the parental cell line U2932 MPA Vector and (***) p=0.0001 for U2932 EBNA2 CL-1.

FIG. 12A-B show that miR-34a over-expression in EBNA2 transfected U2932 cells affects expression of target genes and apoptosis. FIG. 12A: P21 is induced and BCL-2 is downregulated by miR-34a. The expression of these two proteins was tested in miR-34a transfected U2932 and EBNA2 expressing clone. Indeed, p21 was induced and bcl2 was downregulated at 48 h post-transfection of miR-34a. FIG. 12B: Either mimic control or miR-34a mimic transfected U2932 MPA vector and U2932 EBNA2, were stained for apoptosis according to APC Annexin V Apoptosis Detection kit. The detection of the percentage of early apoptotic (only Annexin V), late apoptotic (both Annexin V/PI), and necrotic cells (only PI) was done at 24 and 48 h post transfection, by using Gallios flow analyzer (Beckman Coulter) and the data were analyzed with Kaluza for Gallios Software. One representative experiment of four is shown.

FIG. 13A-B shows the verification of miR-34a transfection in stimulator cells and activation of effector T cells used in standard MLR. FIG. 13A shows miR-34a biosensor was transfected together with either mimic control or miR-34 mimic. Reduction in luciferase activity in U2932 MPA vector and U2932 EBNA2 cells confirmed successful expression of miR-34a. Cells were transfected in triplicates. Error bars represent SEM; (****) p<0.0001. FIG. 13B shows the activation of effector T cells within a PBMC population isolated from two different healthy donors, donor A and donor B, was verified by expression of PD-1 on both CD4+ and CD8+ T cells by flow cytometry.

FIG. 14 is a representative schematic representation of a microfluidic platform for 3D mixed lymphocyte culture. The 3D model devices were seeded with U2932 EBNA2 cells transduced with either miR-34a containing PLL3.7 lentivirus or the corresponding vector control, carrying the GFP marker. 24 hours later, activated T cells were added and after another 48 hours, the devices were processed for caspase-3 staining. The cross section indicates how the cells were placed inside the devices.

FIG. 15 shows detection of ICOSL by flow cytometry in U2932 and its EBNA2 expressing derivatives, demonstrating the downregulation of ICOSL and upregulation of miR-129-5p in EBNA2 transfected DLBCL.

FIG. 16 shows a non-limiting schematic of a mechanism of the present RNA aided immunotherapeutics strategy to treat EBV associated cancer, using an miRNA and anti-PD-L1 (i.e., an antibody or antibody format specific for PD-L1).

FIG. 17A and FIG. 17B are confocal microscope images (FIG. 17A) and a bar graph (FIG. 17B) showing that tumor immunogenicity is enhanced by combining miR-34a and anti-PD-L1 antibody. In FIG. 17A, using the three dimensional biomimetic microfluidic coculture device, U2932 EBNA2 cells were transduced with: (1) either an miR-34a containing pLL3.7 lentivirus, or an anti-PD-L1 antibody and pLL3.7 lentivirus, and cocultivated with activated T cells; (2) both an miR-34a and an anti-PD-L1 antibody with pLL3.7 lentivirus and cocultivated with activated T cells; or (3) the corresponding vector control carrying the GFP marker, and cocultivated with activated T cells. The cocultivation of each of the experiments, i.e., (1) the target miR-34a transduced U2932 EBNA2 cells with activated T cells, (2) the target anti-PD-L1 transduced U2932 EBNA2 cells with activated T cells, (3) the target miR-34a and anti-PD-L1 transduced U2932 EBNA2 cells with activated T cells, and (4) the vector control carrying the GFP marker was carried out for 48 hours before immunostaining. miR-34a containing lentivirus transduced U2932 EBNA2 pLL3.7 cells were stained with: (1) anti-GFP antibody (green; second panel); (2) activated CD8/CD4 T cells were stained with anti-CD8 and anti-CD4 (magenta; fourth panel); (3) apoptotic U2932 EBNA2 pLL3.7 cells were visualized with anti-caspase-3 antibody (red; third panel); and (4) nuclei were counterstained with DAPI (blue; first panel). The overlap of miR-34a transduced U2932 EBNA2 pLL3.7 cells (GFP positive) and caspase-3 (red-third panel) indicates tumor cell death (merged images, pink, fifth panel). Anti-PD-L1 containing lentivirus transduced U2932 EBNA2 pLL3.7 cells were stained with: (1) anti-GFP antibody (green; second panel); (2) activated CD8/CD4 T cells were stained with anti-CD8 and anti-CD4 (magenta; fourth panel); (3) apoptotic U2932 EBNA2 pLL3.7 cells were visualized with anti-caspase-3 antibody (red; third panel); and (4) nuclei were counterstained with DAPI (blue; first panel). The overlap of anti-PD-L1 transduced U2932 EBNA2 pLL3.7 cells (GFP positive) and caspase-3 (red-third panel) indicates tumor cell death (merged images, pink, fifth panel). Both the miR-34a and an anti-PD-L1 containing lentivirus transduced U2932 EBNA2 pLL3.7 cells were stained with: (1) anti-GFP antibody (green; second panel); (2) activated CD8/CD4 T cells were stained with anti-CD8 and anti-CD4 (magenta; fourth panel); (3) apoptotic U2932 EBNA2 pLL3.7 cells were visualized with anti-caspase-3 antibody (red; third panel); and (4) nuclei were counterstained with DAPI (blue; first panel). The overlap of miR-34a and an anti-PD-L1 transduced U2932 EBNA2 pLL3.7 cells (GFP positive) and caspase-3 (red-third panel) indicates tumor cell death (merged images, pink, fifth panel). In FIG. 17B, arbitrary caspase-3 units were calculated for each experimental condition. Statistically significant caspase-3 positive cells were observed only in miR-34a and anti-PD-L1 transduced EBNA2 expressors; data expressed as mean±SEM; (****) p<0.0001. N=4 fields (3-4 devices for each experimental condition). Shown is one representative experiment out of 4 performed. Statistical analysis was performed with Prism-7 software using unpaired T test.

### DETAILED DESCRIPTION

The present disclosure provides a surprising discovery that EBV infected lymphoma cells express altered levels of immune checkpoint proteins like PD-L1 and ICOS-L and identifies, without wishing to be bound by theory, that one of the nine virally encoded proteins, EBNA2 is sufficient to bring about such changes, which in turn makes a lymphoma cell more tumorigenic and less immunogenic. Furthermore, the disclosure identifies that alteration of Immune checkpoint by EBNA2 is through subversion of cellular miRNA expression and in particular, downregulation miR-34a (which targets PD-L1) and upregulation of miR-129 (which downregulates ICOSL).

In one aspect, the present disclosure provides a combination for treating EBV-related cancer, comprising administering an effective amount of one or more of (a) an agent that increases an amount of miR-34a in the subject and (b) an agent that decreases an amount of miR-129 and an immune checkpoint immunotherapy.

In some embodiments, the present disclosure provides a combination comprising an immune checkpoint therapy and an miRNA, and/or a method of using the combination to treat diseases, such as those the cause of which can be influenced by modulating anti-tumor T-cell activity and immune evasion, e.g., EBV-related cancer.

In some embodiments, the present methods synergistically activate immune responses against tumor cells resulting in reduced cancer recurrence and improved cancer treatments.

The combinations and methods disclosed herein are suitable for treating cancer or inhibiting cancer cell proliferation, such as EBV-associated Lymphomas. In some embodiments, the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), and adult T-cell leukemia (ATL)/lymphoma.

Administration of miRNA, such an effective amount of one or more of (a) an agent that increases an amount of miR-34a in the subject and (b) an agent that decreases an amount of miR-129 and an immune checkpoint immunotherapy, reduces EBV-related cancer recurrence.

Among Non-Hodgkin lymphoma (NHL), more than 95% of endemic BLs are associated with EBV. Diffuse large B-cell lymphomas (DLBCLs) constitute about 30% of all NHLs, of which about 10% are EBV associated in immunocompetent patients. Its high frequency makes DLBCL one of the most common cancers in adults. It is noteworthy that the annual global number of cases of EBV positive DLBCLs supersede the total number of BLs. Additionally, EBV is the cause of lymphomas arising in immunocompromised individuals such as AIDS and transplant patients. This suggests that EBV's ability to cause cancer lies in its capacity to evade host immune surveillance. EBV generally establishes one of the following four forms of latency, depending upon the phenotype and the transcription factor repertoire of the infected cells. A complete lack of any virally encoded latent gene expression program as that seen in the resting memory B cell is called latency 0. The expression of the virally encoded EBNA1 and EBERs represents type I latency.

EBV-infected normal B lymphocytes express type I latency in vivo. Under pathological conditions, the viral latent-gene expression varies in different tumors. The phenotypically representative BL and corresponding cell lines express EBNA-1 and LMP2A. When these lines drift towards an immunoblastic phenotype, the viral gene expression is expanded to all growth transformation proteins, EBNA1 to -6 and LMP1, -2A, and -2B. Collectively, this is known as the type III program. The viral latent-gene expression observed in NPC and Hodgkin lymphoma is of intermediate type II latency (LMP1+ EBNA2−). The ability of EBV to transform normal B lymphocytes into permanently growing lymphoblastoid cell lines (LCLs) is attributed to its latent proteins. Among these, LMP1 and EBNA2 have been extensively studied. In particular, it is known that EBNA2 is sine qua non for the virus to transform B cells. Indeed, in keeping with its importance in transformation, EBNA2 expression ensues early after EBV infects naive B cells. This viral protein is also a potent activator of transcription such as CD23 and C-myc but can also negatively regulate genes like BCL6 and Ig. It is a functional homologue of 98 intracellular (Ic) Notch, although they are not interchangeable. It does not bind directly to DNA but activates transcription of many target genes by binding to the transcription factor, RBP-Jk. EBNA2 colocalizes with another B cell specific DNA binding transcription factor, EBF-1, which is essential for the commitment and maintenance of B cell transcription program.

Based on gene expression, DLBCLs are divided into two broad categories, the germinal center (GC) type and the activated B cell type (ABC) or the non-GC type. The overall survival rates in the non-GC (ABC) DLBCL patients, is poor. EBV is associated more frequently with the non-GC DLBCLs, which generally express high levels of PD-L1. Both, EBV associated and high PD-L1 expressing non-GC DLBCLs, have a very poor prognosis. In other hematological malignancies, like Hodgkin Lymphoma (HL), high PD-L1 expression has been reported due to either selective amplification of the PD-L1 locus on chromosome 9p24.1 or EBV infection. These two modes of PD-L1 upregulation are mutually exclusive. It was also shown that LMP1 expression induced PD-L1 promoter activity in B cells. In addition, more than 70% of post-transplant lymphoproliferative disorders, of which EBV is the cause, express PD-L1. In DLBCL, it has been observed that PD-L1 expression is positively correlated with EBV's presence in ABC type DLBCL

Although the presence of EBV is correlated with higher expression of PD-L1 both in HL and DLBCLs, it is not clear if and how the virus is responsible for an increased PD-L1 expression and if this applies to other lymphomas like BLs, as well. While LMP1 has been implicated in induction of PD-L1 in HEK293 cells or in epithelial cells, it is not known if other EBV encoded genes like EBNA2, can regulate PD-L1 in a more frequent cellular setting and natural reservoir for EBV, such as B cells. In this study, we set out to investigate if EBNA2, which is indispensable for EBV's ability to transform B cells, has any effect on PD-L1 and if this involves regulation of cellular miRNAs.

Notwithstanding any theory, miR-34a downregulation by EBNA2 likely involves recruitment of EBF1 at the miR-34a promoter. It has been shown that EBF1 interacts with the N-terminal portion of EBNA2 in a B cell specific manner and this interaction promotes EBNA2 access to chromatin, without involving RBPJk, a known EBNA2-DNA anchor. Analysis of EBNA2 ChIP-Seq datasets from GEO database (accession number: GSM2039170) revealed that EBNA2 peaks at the miR-34a promoter. Furthermore, the data showing the importance of EBF1 in miR-34a regulation by EBNA2 is consistent with previous suggestion that EBNA2 and EBF1 are colocalized at EBNA2 peaks. Recently it was also shown that Ten-Eleven translocation 2 (TET2) is highly expressed in latency III (EBNA2+) BLs and ABC DLBCLs. Interestingly, EBNA2 colocalizes with both EBF1 and TET2. From the results, the role of EBF1 in negative regulation of miR-34a is evident but the possibility that EBNA2 could influence PD-L1 by affecting TET2 needs further investigation. Overall, the present disclosure provides data to support the notion that EBNA2/EBF1 involvement in miR-34a regulation can be therapeutically harnessed for DLBCL and particularly for the drug resistant cases.

EBNA2 is the main driver of B cell transformation induced by EBV. To this end, it is noteworthy that c-MYC is directly unregulated by EBNA2. Additionally, EBNA2 is also a functional homogue of activated Notch. Both c-MYC and activated Notch are known for their oncogenic properties. Most interestingly, both these proteins are miR-34a targets. Based on the data disclosed herein, it is surmised that EBNA2 may not only be the functional homologue of Notch but indeed, it may help keep Notch expression up through downregulation of miR-34a. Casey et al have recently shown that c-MYC can induce PD-L1 expression. Further studies will be required to understand if EBNA2, by downregulating miR-34a increases c-MYC, which in turn may upregulate PD-L1. At present, it is not known if activated Notch genes like c-MYC, has any effect on PD-L1 expression. Based on our data, this exciting possibility needs further investigation.

Increased tumorigenicity is often combined with poor immunogenicity in cancer. Thus, the double edged sword like function of EBNA2 to downregulate miR-34a through EBF1 and consequently upregulate PD-L1, adds to the long list of its oncogenic attributions. To argue against its relevance, because EBNA2 expression is a rarity in lymphomas, would be mistaken, particularly, if wider implications of these findings are considered. EBV induced immunoblastomas of immunocompromised patients, such as in AIDS and transplant, are EBNA2 expressors. A significant proportion of cases within EBV positive ABC DLBCLs are also EBNA2 positive. The viral gene expression pattern in these tumors resembles that of in vitro transformed LCLs and cellular proliferation in both these cell types is indeed EBNA2 driven. Clearly, in patients with compromised T cell immune responses, therapeutic approaches like inactivation of EBNA2 by Crispr-Cas9 gene editing and/or therapeutic introduction of miR-34a mimics will have to be considered.

Epstein-Barr virus (EBV)

The Epstein-Barr virus (EBV), also called human herpesvirus 4 (HHV-4), is one of eight known human herpesvirus types in the herpes family, and is one of the most common viruses in humans. Viruses, being obligate parasites, are under constant pressure to survive in the face of strong host immune responses. To maintain a replicative advantage, they use multiple strategies to make themselves immunologically invisible. This includes downregulation of HLA class I, class II molecules, interference with peptide transport mechanisms, inhibition of proteolysis etc. In this regard, akin to many other viruses, EBV also employs several mechanisms to circumvent immune eradication to establish latency. EBV positive DLBCLs are high PD-L1 expressors and this is confirmed here. However, which virally encoded proteins could be delegated with this task and how do they achieve it, has not been fully explored.

The present disclosure provides a first report of how EBV, through its most critical transformation associated protein, EBNA2, affects PD-L1 expression both in DLBCLs and BLs, by downregulating miR-34a through recruitment of EBF1 to its promoter.

Most EBV positive DLBCLs are non-GC type and high PD-L1 expressors. But it is not known if EBV directly infects a non-GC DLBCL or whether it actually could turn a GC DLBCL into a relatively activated DLBCL. The data of the present disclosure shows strong upregulation of PD-L1 in two in vitro infected GC DLBCLs, which suggests that EBV indeed has the ability to turn a GC derived DLBCL into at least a partially activated one. It is important to clarify here that U2932, often described in the literature as ABC type, is a high BCL6, a hallmark of GC phenotype, expressing cell line (14). Furthermore, a recent detailed classification study suggests that BCL6 is critical marker of GC DLBCL category. Additionally, most ABC DLBCLs express PD-L1. In contrast GC DLBCLs are often PD-L1 negative. U2932 DLBCL is indeed PD-L1 negative. Based on this, we consider U2932 more as an intermediate phenotype DLBCL. Patients with non-GC or activated DLBCLs have both poor prognosis and overall survival rate. Results from our clinical DLBCL samples suggest that EBV positive non-GC DLBCLs have slightly higher PD-L1 expression than those non-GC DLBCLs without the virus. A quantitative IHC image algorithm analysis on digitalized slides revealed that in EBNA2 positive ABC DLBCL samples PD-L1 expression and the staining intensity was higher. Clearly, the effect of EBNA2 alone on PD-L1 would be impossible to determine in clinical samples because EBNA2 alone latency doesn't occur in any tumor associated with EBV. But, notwithstanding the small cohort, the data from clinical samples confirm the in vitro data. Overall, the results are consistent with the suggestion that EBNA2 positive lymphomas may have a better therapeutic outcome with IC blockers. In the first ever use of a microfluidic chip for EBV associated lymphoma growth in 3D, results further show that EBNA2 expressing DLBCLs are less immunogenic. Reconstitution of miR-34a in U2932 EBNA2 cells increased their immunogenicity as seen by IFN-γ production in MLRs and increased apoptosis as measured by caspase-3 expression in Tumor-T cell 3D cocultures.

The 3D biomimetic microfluidic devices, described here for the first time to test immunogenicity of lymphoma cells, provide a quick and economically viable alternative to a more expensive and cumbersome, humanized mouse-based approaches for human tropic viruses like EBV. In addition, these devices might also prove useful in testing the efficacy of combinatorial immunotherapy agents, in lieu of humanized mice. In some embodiments, inactivation of EBNA2 is by Crispr-Cas9 gene editing. In some embodiments, inactivation of EBNA2 is by therapeutic introduction of miR-34a mimics.

### MicroRNAs

MicroRNAs (miRNA or miR) are nucleic acid molecules that are able to regulate the expression of target genes. See review by Carrington et al. Science, Vol. 301(5631):336-338, 2003). MiRNAs are typically short (usually 18-24 nucleotides) and act as repressors of target mRNAs by promoting their degradation, when their sequences are perfectly complementary, and/or by inhibiting translation, when their sequences contain mismatches. Notwithstanding any theory, mature miRNAs are believed to be generated by RNA polymerase II (pol II) or RNA polymerase III (pol III; see Qi et al., (2006) Cellular & Molecular Immunology, Vol. 3:411-419) and arise from initial transcripts termed primary miRNA transcripts (pri-miRNAs). These pri-miRNAs are frequently several thousand bases long and are therefore processed to make the much shorter mature miRNAs. This processing is believed to occur in two steps. First, pri-miRNAs are processed in the nucleus by the RNase Drosha into about 70- to about 100-nucleotide hairpin-shaped precursors (pre-miRNAs). Second, after transposition to the cytoplasm, the hairpin pre-miRNAs are further processed by the RNase Dicer to produce a double-stranded miRNA. A mature miRNA strand is then incorporated into the RNA-induced silencing complex (RISC), where it associates with its target mRNA by base-pair complementarity and leads to suppression of protein expression.

In some embodiments, miRNA genes may be located within introns of protein-coding genes or within introns or exons of noncoding transcriptional units. The expression of intronic miRNAs may coincide with that of the hosting transcriptional units because they are typically oriented in the same direction and are coordinately expressed with the pre-mRNAs in which they reside. In some embodiments, miRNAs may bind to sequences within the 3′ untranslated region (3′UTR) of target gene transcripts. In some embodiments, miRNAs may bind to sequences outside of the 3′UTR of target gene transcripts. In some embodiments, miRNAs may bind to both within and outside the 3′UTR of target gene transcripts.

In some embodiments, nucleotide pairing between the second and seventh nucleotides of the miRNA (the miRNA seed sequence) and the corresponding sequence along the target 3′UTR (seed match) may occur for target recognition. Accordingly, the binding between miRNA and target may comprise about a 5 nucleotide base pairing. Additionally, the binding between miRNA and target may comprise more than a 5 nucleotide base pairing. In some embodiments, the binding between an miRNA and the gene that it regulates may be mediated by the miRNA binding up to 2, up to 4, up to 6, up to 8, or up to 10 sites of the target nucleic acid.

MiRNAs of the present disclosure may regulate nucleic acids, including but not limited to cell proliferative genes such as genes of a marker linked to a cancer by direct binding. This binding may be perfectly complementary to the target nucleic acid or contain mismatches. The effect of this binding may be to promote degradation and/or to inhibit translation of the target.

### MiR-34a

The miR-34 family members are transcriptionally induced by p53. They suppress transcription of genes important in cell cycle progression, anti-apoptotic functions, and regulation of cell growth. Expression of miRNAs is altered in a broad range of cancers, with frequent downregulation of both p53 and miR-34. The latter is downregulated in chronic lymphocytic leukemia (CLL) and acute myeloid leukemia (AML). It is frequently downregulated in a wide variety of cancers. In keeping with this, its expression is often reduced in ABC type of DLBCL cell lines and tumor tissues. Overall survival of those patients with low miR-34a is poorer and over-expression of miR-34a in ABC DLBCL lines, make them responsive to doxorubicin treatment. EBNA2 downregulation of miR-34a are consistent with the reported lower expression of miR-34a in ABC DLBCLs and doxorubicin resistance. Indeed, in Lat III ABC DLBCLs, EBNA2 might contribute to chemoresistance and poor prognosis by downregulating miR-34a. Additionally, it has been shown that intravenous delivery miR-34a treatment of mice with U2932 DLBCL xenografts suppresses tumor growth, thus underpinning its therapeutic utility. Among its noted targets is the oncogene FOXP1. Interestingly, the IC protein, PD-L1, has been shown to be a validated target of miR-34a. In AML, miR-34a targets PD-L1. Results show that EBV, through its growth transformation associated protein EBNA2, increases PD-L1 by downregulating miR-34a. Furthermore, in the presence of EBNA2, pre-miR-34a and miR-34a promoter activity is reduced and this suggests that EBNA2 affects miR-34a transcription. Notwithstanding any theory, miR-34a downregulation by EBNA2 likely involves recruitment of EBF1 at the miR-34a promoter. In some embodiments, the present disclosure treats or prevents EBV-related cancer in a subject through the upregulation of miRNAs, such as miR-34a. In some embodiments, an agent that increases an amount of miR-34a is selected from one or more of miR-34a, or miR-34a mimetic. In some embodiments, an agent that increases an amount of miR-34a is an inhibitor of EBF1. In some embodiments, the nucleic acid encoding mir-34a comprises or consists of UGGCAGUGUCUUAGCUGGUUGU (SEQ ID NO: 11).

### MiR-129

microRNA-129 (miR-129-5p) has been shown to trigger apoptosis by suppressing a key anti-apoptotic protein, B-cell lymphoma 2 (BCL2). Ectopic expression of miR-129 is shown to promote apoptosis, inhibit cell proliferation and cause cell-cycle arrest. In vitro data suggest that ICOS-L is downregulated by EBNA2 by upregulation of miR-129-5p and other ICOSL targeting miRNAs. In some embodiments, the nucleic acid encoding mir-129-5p comprises or consists of CUUUUUGCGGUCUGGGCUUGC (SEQ ID NO: 12). In some embodiments, an agent that decreases an amount of miR-129 is selected from one or more of an antisense oligonucleotide, an antagomir or a construct expressing a miRNA inhibitor.

### Antisense Inhibitors

An inhibitor of miRNA includes an antisense oligonucleotide, antagomiR or a construct expressing a miRNA inhibitor. Antisense oligonucleotides can include ribonucleotides or deoxyribonucleotides or a combination thereof. Antisense oligonucleotides may have at least one chemical modification (non-limiting examples are sugar or backbone modifications). For instance, suitable antisense oligonucleotides can be comprised of one or more conformationally constrained or bicyclic sugar nucleoside modifications (BSN) that confer enhanced thermal stability to complexes formed between the oligonucleotide containing BSN and their complementary miRNA target strand. For example, in one embodiment, the antisense oligonucleotides contain at least one locked nucleic acid. Locked nucleic acids (LNAs) contain a 2′-O, 4′-C-methylene ribonucleoside (structure A) wherein the ribose sugar moiety is in a locked conformation. In another embodiment, the antisense oligonucleotides contain at least one 2′, 4′-C-bridged 2′ deoxyribonucleoside (CDNA, structure B). See, e.g., U.S. Pat. No. 6,403,566 and Wang et al., (1999) Bioorganic and Medicinal Chemistry Letters, Vol. 9: 1147-1150, both of which are herein incorporated by reference in their entireties. In yet another embodiment, the antisense oligonucleotides contain at least one modified nucleoside having the structure shown in structure C. The antisense oligonucleotides targeting miRNAs that regulate tumor suppressors can contain combinations of BSN (LNA, CDNA, and the like) or other modified nucleotides, and ribonucleotides or deoxyribonucleotides.

Alternatively, the antisense oligonucleotides can comprise peptide nucleic acids (PNAs), which contain a peptide-based backbone rather than a sugar-phosphate backbone. Other modified sugar or phosphodiester modifications to the antisense oligonucleotide are also contemplated. By way of non-limiting examples, other chemical modifications can include 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages (see, e.g., U.S. Pat. Nos. 6,693,187 and 7,067,641, which are herein incorporated by reference in their entireties). In one embodiment, antisense oligonucleotides targeting oncogenic miRNAs contain 2′-O-methyl sugar modifications on each base and are linked by phosphorothioate linkages. Antisense oligonucleotides, particularly those of shorter lengths (e.g., less than 16 nucleotides, 7-8 nucleotides) can comprise one or more affinity enhancing modifications, such as, but not limited to, LNAs, bicyclic nucleosides, phosphonoformates, 2′ O-alkyl modifications, and the like. In some embodiments, suitable antisense oligonucleotides are 2′-O-methoxyethyl gapmers which contain 2′-O-methoxyethyl-modified ribonucleotides on both 5′ and 3′ ends with at least ten deoxyribonucleotides in the center. These gapmers are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. For instance, and not intending to be limiting, to facilitate in vivo delivery and stability, the antisense oligonucleotide can be linked to a steroid, such as cholesterol moiety, a vitamin, a fatty acid, a carbohydrate or glycoside, a peptide, or other small molecule ligand at its 3′ end.

In some embodiments, antisense oligonucleotides useful for inhibiting the activity of miRNAs are about 5 to about 25 nucleotides in length, about 10 to about 30 nucleotides in length, or about 20 to about 25 nucleotides in length. In certain embodiments, antisense oligonucleotides targeting oncogenic miRNAs are about 8 to about 18 nucleotides in length, in other embodiments about 12 to about 16 nucleotides in length, and in other embodiments about 7-8 nucleotides in length. Any 7-mer or longer complementary to an oncogenic miRNA may be used, i.e., any anti-miR complementary to the 5′ end of the miRNA and progressing across the full complementary sequence of the miRNA.

Antisense oligonucleotides can comprise a sequence that is at least partially complementary to a mature or minor (i.e., star) oncogenic miRNA sequence, e.g., at least about 75%, 80%, 85%, 90%, 95%, 96%, 97%, 98%, or 99% complementary to a mature or minor (i.e. star) oncogenic miRNA sequence. In some embodiments, the antisense oligonucleotide can be substantially complementary to a mature or minor oncogenic miRNA sequence, that is at least about 90%, 95%, 96%, 97%, 98%, or 99% complementary to a target polynucleotide sequence. In one embodiment, the antisense oligonucleotide comprises a sequence that is 100% complementary to a mature or minor oncogenic miRNA sequence.

As used herein, substantially complementary refers to a sequence that is at least about 95%, 96%, 97%, 98%, 99%, or 100% complementary to a target polynucleotide sequence (non-limiting examples are mature, minor, precursor miRNA, or pri-miRNA sequence).

In some embodiments, the antisense oligonucleotides are antagomirs. Antagomirs are single-stranded, chemically-modified ribonucleotides that are at least partially complementary to miRNAs and therefore may silence them. See, e.g., Krtitzfeldt, et al. Nature (2005) 438 (7068): 685-9. Antagomirs may comprise one or more modified nucleotides, such as 2′-O-methyl-sugar modifications. In some embodiments, antagomirs comprise only modified nucleotides. Antagomirs can also comprise one or more phosphorothioate linkages resulting in a partial or full phosphorothioate backbone. To facilitate in vivo delivery and stability, the antagomir can be linked to a cholesterol or other moiety at its 3′ end. Antagomirs suitable for inhibiting can be about 15 to about 50 nucleotides in length, about 18 to about 30 nucleotides in length, and about 20 to about 25 nucleotides in length. The antagomirs can be at least about 75%, 80%, 85%, 90%, 95%, 96%, 97%, 98%, or 99% complementary to a mature or minor oncogenic miRNA sequence. In some embodiments, the antagomir may be substantially complementary to a mature or minor oncogenic miRNA sequence, that is at least about 95%, 96%, 97%, 98%, or 99% complementary to a target polynucleotide sequence. In other embodiments, the antagomirs are 100% complementary to a mature or minor oncogenic miRNA sequence.

Antisense oligonucleotides or antagomirs may comprise a sequence that is substantially complementary to a precursor miRNA sequence (pre-miRNA) or primary miRNA sequence (pri-miRNA) of an oncogenic miRNA. In some embodiments, the antisense oligonucleotide comprises a sequence that is located outside the 3′-untranslated region of a target of that miRNA. In some embodiments, the antisense oligonucleotide comprises a sequence that is located inside the 3′-untranslated region of a target of that miRNA.

Any of the inhibitors or agonists of the oncogenic miRNAs described herein can be delivered to a target cell (a non-limiting example is a cancer cell) by delivering to the cell an expression vector encoding the miRNA inhibitors or agonists. A vector is a composition of matter which can be used to deliver a nucleic acid of interest to the interior of a cell. Numerous vectors are known in the art, including, but not limited to, linear polynucleotides, polynucleotides associated with ionic or amphiphilic compounds, plasmids, and viruses. Thus, the term vector includes an autonomously replicating plasmid or a virus. Examples of viral vectors include, but are not limited to, adenoviral vectors, adeno-associated virus vectors, retroviral vectors, and the like. An expression construct can be replicated in a living cell, or it can be made synthetically. For purposes of this application, the terms expression construct, expression vector, and vector are used interchangeably to demonstrate the application of the invention in a general, illustrative sense, and are not intended to limit the invention.

In some embodiments, an expression vector for expressing an inhibitor of an oncogenic miRNA comprises a promoter operably linked to a polynucleotide encoding an antisense oligonucleotide. The sequence of the expressed antisense oligonucleotide may be partially or perfectly complementary to a mature or minor sequence of an oncogenic miRNA. The phrase operably linked or under transcriptional control as used herein means that the promoter is in the correct location and orientation in relation to a polynucleotide to control the initiation of transcription by RNA polymerase and expression of the polynucleotide.

As used herein, a promoter refers to a DNA sequence recognized by the synthetic machinery of the cell, or introduced synthetic machinery, required to initiate the specific transcription of a gene. Suitable promoters include, but are not limited to, RNA pol I, pol II, pol III, and viral promoters (e.g., human cytomegalovirus (CMV) immediate early gene promoter, the SV40 early promoter, and the Rous sarcoma virus long terminal repeat). In one embodiment, the promoter is a tissue-specific promoter, such as, by way of non-limiting example, the prostate-specific Probasin promoter ARR2PB. In certain embodiments, the promoter operably linked to a polynucleotide encoding an miRNA inhibitor or a polynucleotide encoding a tumor-suppressor regulating miRNA can be an inducible promoter. Inducible promoters are known in the art and include, but are not limited to, the tetracycline promoter, the metallothionein IIA promoter, the heat shock promoter, the steroid/thyroid hormone/retinoic acid response elements, the adenovirus late promoter, and the inducible mouse mammary tumor virus LTR. Methods of delivering expression constructs and nucleic acids to cells are known in the art and can include, by way of non-limiting example, calcium phosphate co-precipitation, electroporation, microinjection, DEAE-dextran, lipofection, transfection employing polyamine transfection reagents, cell sonication, gene bombardment using high velocity microprojectiles, and receptor-mediated transfection.

The present invention also includes scavenging or clearing inhibitors of oncogenic miRNAs following treatment. Scavengers may include isolated nucleic acids that are complementary to miRNA inhibitors or vectors expressing the same. Therefore, they may bind to miRNA inhibitors or vectors expressing the same and, in doing so, prevent the binding between miRNA and target. The method may comprise overexpressing binding sites for the tumor suppressive inhibitors in a tissue.

### Immune Checkpoint Genes

Immune checkpoints (IC) regulate T cell responses to maintain self-tolerance. They deliver costimulatory and coinhibitory signals to T cells (20). PD-L1, mainly expressed by antigen presenting cells engages its receptor PD-1 on T cells, to provide a growth inhibitory signal. Different tumors express high PD-L1 to evade immune recognition and consistently, inhibition of PD-1/PD-L1 and other IC molecules have become important targets of cancer immunotherapy. In some embodiments, the immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

### Programmed Cell Death-1 (PD-1)

PD-1 is a cell surface receptor that is a member of the CD28 family of T-cell regulators, within the immunoglobulin superfamily of receptors. The human PD-1 gene is located at chromosome 2q37, and the full-length PD-1 cDNA encodes a protein with 288 amino acid residues with 60% homology to murine PD-1. It is present on CD4− CD8− (double negative) thymocytes during thymic development and is expressed upon activation in mature hematopoietic cells such as T and B cells, NKT cells and monocytes after prolonged antigen exposure.

Notwithstanding any theory, it is contemplated that binding of the ligand PD-L1 to PD-1 downregulates effector anti-tumor T-cell activity and facilitates immune evasion. This is supported by the finding of an association between PD-1/PD-L1 expression and poor prognosis in several tumor types including gastric, ovarian, lung and renal carcinomas. PD-1 has been reported to be predominantly expressed by tumor infiltrating T lymphocytes, EBV-associated tumors. Notwithstanding any theory, it is contemplated that targeting PD-1 may act as an effective therapeutic strategy for cancer. The principal method for targeting PD-1 clinically has been through the development of genetically engineered monoclonal antibodies that inhibit either PD-1 or PD-L1 function.

### Programmed Death-Ligand (PD-L1 and PD-L2)

PD-L1 has also been shown to bind to B7-1 (CD80), an interaction that also suppresses T-cell proliferation and cytokine production. Cancer cells drive high expression levels of PD-L1 on their surface, allowing activation of the inhibitory PD-1 receptor on any T cells that infiltrate the tumor microenvironment, effectively switching those cells off. Indeed, upregulation of PD-L1 expression levels has been demonstrated in many different cancer types (e.g., EBV-associated tumors), and high levels of PD-L1 expression have been linked to poor clinical outcomes. In some embodiments, the subject is undergoing treatment with an immune checkpoint immunotherapy selected from an agent that modulates PD-L1. In some embodiments, the subject is undergoing treatment with an immune checkpoint immunotherapy selected from an agent that modulates PD-L2.

### Inducible T-Cell Costimulator Ligand (ICOSL) and Inducible T-Cell Costimulator Ligand (ICOSL)

ICOS is an inducible T cell costimulatory receptor molecule that displays some homology to CD28 and CTLA-4, and interacts with B7-H2 expressed on the surface of antigen-presenting cells. ICOS has been implicated in the regulation of cell-mediated and humoral immune respons-es. The present disclosure provides in vitro data showing that EBNA2 can simultaneously upregulate IC inhibitor PD-L1 and downregulate IC costimulator ICOS-L. The RNA seq and other in vitro data suggest that EBNA2 increases PD-L1 by downregulation miR-34a. Conversely, ICOS-L is downregulated by EBNA2 by upregulation of miR-129-5p and other ICOSL targeting miRNAs. Additionally, immunogenicity of EBV-infected tumors is increased by upregulating ICOSL protein by downregulating ICOSL targeting miRNAs which were found to be upregulat-ed in EBV-infected lymphoma.

In some embodiments, the immune-modulating agent targets one or more immune checkpoint genes including for example, PD-1, PD-L1, and PD-L2. In some embodiments, the immune-modulating agent is PD-1 inhibitor. In some embodiments, the immune-modulating agent is an antibody or antigen binding fragment thereof, specific for one or more of PD-1, PD-L1, and PD-L2. For instance, in some embodiments, the immune-modulating agent is an antibody or antigen binding fragment thereof such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL328OA (ROCHE). In some embodiments, the immune-modulating agent targets one or more of CD137 or CD137L. In some embodiments, the immune-modulating agent is an antibody or antigen binding fragment thereof specific for one or more of CD137 or CD137L. For instance, in some embodiments, the immune-modulating agent is an antibody or antigen binding fragment thereof such as, by way of non-limitation, urelumab (also known as BMS-663513 and anti-4-1BB antibody). In some embodiments, the present agent that increases an amount of miR-34a in the subject and/or agent that decreases an amount of miR-129 is combined with urelumab (optionally with one or more of nivolumab, lirilumab, and urelumab) for the treatment of solid tumors and/or B-cell non-Hodgkins lymphoma and/or head and neck cancer and/or multiple myeloma. In some embodiments, the immune-modulating agent is an agent that targets one or more of CTLA-4, AP2M1, CD80, CD86, SHP-2, and PPP2R5A.

### Cytotoxic T-Lymphocyte-Associated Protein 4 (CTLA-4)

CTLA-4 is a protein receptor that, functioning as an immune checkpoint, downregulates immune responses. CTLA4 is constitutively expressed in regulatory T cells but only upregulated in conventional T cells after activation—a phenomenon which is particularly notable in cancers. In some embodiments, the subject is undergoing treatment with an immune checkpoint immunotherapy selected from an agent that modulates CTLA-4.

### Cancer

Cancer is a group of diseases characterized by uncontrolled cell division which can lead to abnormal tissue and, in turn, disruption of normal physiologic processes and, possibly, death. Cancers have various etiologies and may be responsive to agents that affect aspects of these etiologies. For example, a reduction or loss of nucleic acids that are linked to cancer development may prove fruitful in the treatment of various cancers, including blood-based cancers and breast cancers. Such treatments may replace or supplement existing treatments. Therefore, there is a need in the art for treatment methods for cancers, including blood-based cancers, such as Non-Hodgkin lymphoma (NHL), that target miRNAs that bind to cancer related genes. Further, there is a need for agents designed to this end which can be produced cheaply, delivered effectively, and which display adequate bioavailability.

In some embodiments, the present invention encompasses methods of treating or preventing cancer and/or a metastasis in a subject in need thereof. In some embodiments, representative cancers and/or tumors and/or metastases of the present invention include a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome. See, e, g., Weinberg, The Biology of Cancer, Garland Science: London 2006, the contents of which are hereby incorporated by reference. In some embodiments, the cancer to be treated or prevented is a blood-based cancer or related disease including, for example, a lymphoma, leukemia, myeloma or myelodysplastic/myeloproliferative neoplasm (MDS/MPN).

In some embodiments, the cancer is an EBV-related cancer. In some embodiments, the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), adult T-cell leukemia (ATL)/lymphoma and Post-transplant lymphoproliferative disease (PTLD).

In some embodiments, the lymphoma is a Hodgkin lymphoma or a non-Hodgkin lymphoma. In some embodiments, the lymphoma is precursor T-cell leukemia/lymphoma, f, d, mantle cell lymphoma, B-cell chronic lymphocytic leukemia/lymphoma, MALT lymphoma, Burkitt's lymphoma, mycosis fungoides, peripheral T-cell lymphoma-not-otherwise-specified, nodular sclerosis form of Hodgkin lymphoma, or mixed-cellularity subtype of Hodgkin lymphoma.

In some embodiments, Tumor Nymph and Metastasis (TNM), staging system is used to describe the growth and spread of Diffuse Large B-cell lymphomas. In some embodiments, numbers or letters are used after T, N, and M to provide more details about each of these factors. In some embodiments, higher numbers denote a cancer that is more advanced. Once the T, N, and M categories have been determined, this information is combined in a process called stage grouping, to assign an overall stage.

In some embodiments, the T, N, and M categories are combined to assign an overall stage of 0, I, II, III, or IV in a process called stage grouping. The stages identify cancers that have a similar prognosis. Mostly, patients with lower stage numbers tend to have a better prognosis.

In some embodiments, the stage grouping is Stage 0; Tis, N0, M0: The cancer is found only in the layer of cells lining the air passages. It has not invaded other lung tissues nor spread to lymph nodes or distant sites.

In some embodiments, the stage grouping is Stage IA; T1, N0, M0: The cancer is no larger than 3 centimeters, has not spread to the membranes that surround the lungs, does not affect the main branches of the bronchi and has not spread to lymph nodes or distant sites.

In some embodiments, the stage grouping is Stage IB; T2, N0, M0: The cancer is larger than 3 cm, or involves a main bronchus, but is not near the carina or it has spread to the pleura or the cancer is partially clogging the airways. It has not spread to lymph nodes or distant sites.

In some embodiments, the stage grouping is Stage IIA; T1, N1, M0: The cancer is no larger than 3 centimeters, has not spread to the membranes that surround the lungs, does not affect the main branches of the bronchi. It has spread to nearby or hilar lymph nodes, but not too distant sites.

In some embodiments, the stage grouping is Stage IIB; T2, N1, M0 or T3, N0, M0: The cancer is larger than 3 cm, or involves a main bronchus, but is not near the carina or it has spread to the pleura or the cancer is partially clogging the airways. It has spread to nearby or hilar lymph nodes, but not too distant sites, or, it has spread to the chest wall or the diaphragm, the mediastinal pleura, or membranes surrounding the heart, or it invades a main bronchus and is close to the carina or it has grown into the airways enough to cause an entire lung to collapse or to cause pneumonia in the entire lung. It has not spread to lymph nodes or distant sites.

In some embodiments, the stage grouping is Stage IIIA; T1 or 2, N2, M0 or T3, N1 or 2, M0: The cancer can be any size, or involves a main bronchus, but is not near the carina or it has spread to the pleura or the cancer is partially clogging the airways. It has spread to nodes in the middle of the chest (mediastinum), but not too distant sites, or, it has spread to the chest wall or the diaphragm, the mediastinal pleura, or membranes surrounding the heart, or it invades a main bronchus and is close to the carina or it has grown into the airways enough to cause an entire lung to collapse or to cause pneumonia in the entire lung. It has spread to lymph nodes anywhere in the chest on the same side as the cancer, but not too distant sites.

In some embodiments, the stage grouping is Stage IIIB; T1, 2 or 3, N3, M0 or T4, N0, 1, 2 or 3, M0: The cancer can be of any size. It has spread to lymph nodes around the collarbone on either side, or to hilar or mediastinal lymph nodes on the side opposite the cancerous lung or, it has spread to the mediastinum, the heart, the windpipe (trachea), the esophagus (tube connecting the throat to the stomach), the backbone, or the carina or two or more separate tumor nodules are present in the same lobe, or there is a fluid containing cancer cells in the space surrounding the lung. The cancer may or may not have spread to lymph nodes. It has not spread to distant sites.

In some embodiments, the stage grouping is Stage IV; Any T, Any N, M1: The cancer has spread to distant sites.

As used herein, the term subject or patient refers to any vertebrate including, without limitation, humans and other primates (e.g., chimpanzees and other apes and monkey species), farm animals (e.g., cattle, sheep, pigs, goats, and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In some embodiments, the subject is a human.

### Pharmaceutical Compositions

Another embodiment of the present disclosure is a pharmaceutical composition, or use of pharmaceutical composition, comprising an agent that increases an amount of miR-34a in the subject and (b) an agent that decreases an amount of miR-129, an immune checkpoint immunotherapy, and a pharmaceutically acceptable carrier. Where clinical applications are contemplated, pharmaceutical compositions may be prepared in a form appropriate for the intended application. Generally, this will entail preparing compositions that are essentially free of pyrogens, as well as other impurities that could be harmful to humans or animals.

In some embodiments, a pharmaceutical composition comprises an effective dose of an miRNA, by way of non-limiting example, miR-34, and a pharmaceutically acceptable carrier. In some embodiments, a pharmaceutical composition comprises an effective dose of an miRNA, by way of non-limiting example, miR-129, and a pharmaceutically acceptable carrier. An effective dose is an amount sufficient to affect a beneficial or desired clinical result. An effective dose of an miRNA of the disclosure may be from about 1 mg/kg to about 100 mg/kg, about 2.5 mg/kg to about 50 mg/kg, or about 5 mg/kg to about 25 mg/kg. The precise determination of what would be considered an effective dose may be based on factors individual to each patient, including their size, age, type of cancer, and nature of inhibitor or agonist (non-limiting examples include antagomir, expression construct, antisense oligonucleotide, polynucleotide duplex, etc.). Therefore, dosages can be readily ascertained by those of ordinary skill in the art from this disclosure and the knowledge in the art. For example, doses may be determined with reference Physicians' Desk Reference, 66th Edition, PDR Network; 2012 Edition (Dec. 27, 2011), the contents of which are incorporated by reference in its entirety.

A beneficial or desired clinical result may include, inter alia, a reduction in tumor size and/or tumor growth and/or a reduction of a cancer marker that is associated with the presence of cancer as compared to what is observed without administration of the inhibitor. A beneficial or desired clinical result may also include, inter alia, an increased presence of a marker that is associated with a reduction of cancer as compared to what is observed without administration of the inhibitor. Also included in a beneficial or desired clinical result is, inter alia, an increased amount of a gene comprising a marker linked to cancer etiology as compared to what is observed without administration of the inhibitor. The gene comprising a marker linked to cancer etiology may include, for example, EBF1.

### Dosing and Administration

One will generally desire to employ appropriate salts and buffers to render delivery vehicles stable and allow for uptake by target cells. Aqueous compositions of the present invention comprise an effective amount of the delivery vehicle comprising an agent that increases an amount of miR-34a in the subject and (b) an agent that decreases an amount of miR-129, and an immune checkpoint immunotherapy (e.g., liposomes or other complexes or expression vectors) dissolved or dispersed in a pharmaceutically acceptable carrier or aqueous medium. The phrases pharmaceutically acceptable or pharmacologically acceptable refer to molecular entities and compositions that do not produce adverse, allergic, or other untoward reactions when administered to an animal or a human. As used herein, pharmaceutically acceptable carrier includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or polynucleotides of the compositions.

The active compositions of the present invention may include classic pharmaceutical preparations. Administration of these compositions according to the present invention may be via any common route so long as the target tissue is available via that route. This includes oral, nasal, or buccal. Alternatively, administration may be by intratumoral, intradermal, subcutaneous, intramuscular, intraperitoneal or intravenous injection, or by direct injection into cancer tissue. The agents disclosed herein may also be administered by catheter systems. Such compositions would normally be administered as pharmaceutically acceptable compositions as described herein.

Upon formulation, solutions may be administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic with, for example, sufficient saline or glucose. Such aqueous solutions may be used, for example, for intratumoral, intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pages 1035-1038 and 1570-1580, the contents of which are hereby incorporated by reference). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA Office of Biologics standards.

In some embodiments, the first and second agents may be administered in either order (e.g., first then second or second then first) or concurrently.

In some embodiments of the present disclosure includes a method of treating or preventing an EBV-related cancer in a subject in need thereof comprising administering to the subject a first agent comprising (a) an agent that increases an amount of miR-34a in the subject and (b) an agent that decreases an amount of miR-129, an immune checkpoint immunotherapy and a second agent that is or comprises at least one other cancer biologic, therapeutic, chemotherapeutic or drug.

In some embodiments, the immune checkpoint immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

In some embodiments, the immune checkpoint immunotherapy selected from an agent that modulates one or more of PD-1, PD-L1, PD-L2, ICOS, ICOSL, and CTLA-4 can be administered over any suitable period of time, such as a period from about 1 day to about 12 months. In some embodiments, for example, the period of administration can be from about 1 day to 90 days; from about 1 day to 60 days; from about 1 day to 30 days; from about 1 day to 20 days; from about 1 day to 10 days; from about 1 day to 7 days. In some embodiments, the period of administration can be from about 1 week to 50 weeks; from about 1 week to 50 weeks; from about 1 week to 40 weeks; from about 1 week to 30 weeks; from about 1 week to 24 weeks; from about 1 week to 20 weeks; from about 1 week to 16 weeks; from about 1 week to 12 weeks; from about 1 week to 8 weeks; from about 1 week to 4 weeks; from about 1 week to 3 weeks; from about 1 week to 2 weeks; from about 2 weeks to 3 weeks; from about 2 weeks to 4 weeks; from about 2 weeks to 6 weeks; from about 2 weeks to 8 weeks; from about 3 weeks to 8 weeks; from about 3 weeks to 12 weeks; or from about 4 weeks to 20 weeks.

In some embodiments, the immune checkpoint immunotherapy selected from an agent that modulates one or more of PD-1, PD-L1, PD-L2, ICOS, ICOSL, and CTLA-4 can be administered every day, every other day, every week, every 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or every 20 weeks, or every month

In some embodiments, the immune checkpoint immunotherapy selected from an agent that modulates one or more of PD-1, PD-L1, PD-L2, ICOS, ICOSL, and CTLA-4 can be administered at the 0.01 mg/kg per day to about 10 mg/kg per day. In some embodiments, dosages can range from about 0.1 mg/kg, 0.5 mg/kg, 1 mg/kg, 1.5 mg/kg, 3 mg/kg, 5 mg/kg, 6 mg/kg, 7.5 mg/kg, or about 10 mg/kg. In some embodiments, the dose will be in the range of about 0.1 mg/day to about 5 mg/kg; about 0.1 mg/day to about 10 mg/kg; about 0.1 mg/day to about 20 mg/kg; about 0.1 mg to about 30 mg/kg; or about 0.1 mg to about 40 mg/kg or about In In some embodiments, the immune checkpoint immunotherapy selected from an agent that modulates one or more of PD-1, PD-L1, PD-L2, ICOS, ICOSL, and CTLA-4 can be administered at 0.1 mg to about 50 mg/kg or in single, divided, or continuous doses (which dose may be adjusted for the patient's weight in kg, body surface area in m2, and age in years).

In some embodiments, the immune checkpoint immunotherapy selected from an agent that modulates one or more of PD-1, PD-L1, PD-L2, ICOS, ICOSL, and CTLA-4 can be administered at 0.01 mg/kg to about 500 mg/kg, for example, about 0.1 mg/kg to about 200 mg/kg (such as about 100 mg/kg), or about 0.1 mg/kg to about 10 mg/kg (such as about 0.1 mg/kg, 0.5 mg/kg, 1 mg/kg, 1.5 mg/kg, 3 mg/kg, 5 mg/kg, 6 mg/kg, 7.5 mg/kg, or about 10 mg/kg).

In some embodiments, the immune checkpoint immunotherapy agent modulates programmed cell death protein-1 (PD-1). In some embodiments, the agent that modulates PD-1 is an antibody or antibody format specific for PD-1. In some embodiments, the antibody or antibody format specific for PD-1 is selected from Nivolumab, Pembrolizumab, Pidilizumab, BMS-936559, Atezolizumab, or Avelumab. In some embodiments, an antibody or antibody format specific for PD-1 is Nivolumab and can be administered at 240 mg every 2 weeks. In some embodiments, an antibody or antibody format specific for PD-1 is Pembrolizumab and can be administered at 200 mg every 3 weeks. In some embodiments, an antibody or antibody format specific for PD-1 is Pidilizumab and can be administered at 200 mg every 3 weeks. In some embodiments, an antibody or antibody format specific for PD-1 is BMS-936559 and can be administered at 0.1 mg/kg every 2 weeks. In some embodiments, an antibody or antibody format specific for PD-1 is Atezolizumab and can be administered at 1200 mg every 3 weeks. In some embodiments, an antibody or antibody format specific for PD-1 is Avelumab and can be administered at 10 mg/kg every 2 weeks

In some embodiments, the immune checkpoint immunotherapy agent modulates programmed cell death protein-1 (PD-L1). In some embodiments, the agent that modulates PD-L1 is an antibody or antibody format specific for PD-L1. In some embodiments, the antibody or antibody format specific for PD-L1 is selected from Nivolumab, Pembrolizumab, Pidilizumab, BMS-936559, Atezolizumab, or Avelumab. In some embodiments, the antibody or antibody format specific for PD-L1 is Nivolumab and can be administered at 240 mg every 2 weeks. In some embodiments, the antibody or antibody format specific for PD-L1 is Pembrolizumab and can be administered at 200 mg every 3 weeks. In some embodiments, the antibody or antibody format specific for PD-L1 is Pidilizumab and can be administered at 200 mg every 3 weeks. In some embodiments, the antibody or antibody format specific for PD-L1 is BMS-936559 and can be administered at 0.1 mg/kg every 2 weeks. In some embodiments, the antibody or antibody format specific for PD-L1 is Atezolizumab and can be administered at 1200 mg every 3 weeks. In some embodiments, the antibody or antibody format specific for PD-L1 is Avelumab and can be administered at 10 mg/kg every 2 weeks. In some embodiments, the antibody or antibody format specific for PD-L1 is Durvalumab and can be administered at 10 mg/kg every 2 weeks.

In some embodiments, an antibody or antibody format specific for PD-L2 is Nivolumab and can be administered at 240 mg every 2 weeks. In some embodiments, PD-L2 is an antibody or antibody format specific for PD-L2 is Pembrolizumab and can be administered at 200 mg every 3 weeks. In some embodiments, the antibody or antibody format specific for PD-L2 is Pidilizumab and can be administered at 200 mg every 3 weeks. In some embodiments, the antibody or antibody format specific for PD-L2 is BMS-936559 and can be administered at 0.1 mg/kg every 2 weeks. In some embodiments, the antibody or antibody format specific for PD-L2 is Atezolizumab and can be administered at 1200 mg every 3 weeks. In some embodiments, the antibody or antibody format specific for PD-L2 is Avelumab and can be administered at 10 mg/kg every 2 weeks. In some embodiments, the antibody or antibody format specific for PD-L2 is Durvalumab and can be administered at 10 mg/kg every 2 weeks.

In some embodiments, the antibody or antibody format specific for ICOS is JTX-2011 and can be administered as a 0.3 mg/kg every 21 days.

In some embodiments, the antibody or antibody format specific for CTLA-4 is tremelimumab and can administered at 3 mg/kg, 6 mg/kg or 10 mg/kg.

In some embodiments, the antibody or antibody format specific for CTLA-4 is Ipilimumab and can administered at 5 mg/mL 12 weeks.

In some embodiments, the present disclosure includes a method of treating or preventing an EBV-related cancer in a subject in need thereof comprising administering to the subject a first agent comprising (a) an agent that increases an amount of miR-34a in the subject and/or (b) an agent that decreases an amount of miR-129, and a second agent comprising an immune checkpoint immunotherapy.

In some embodiments, the present disclosure includes a method of treating or preventing an EBV-related cancer in a subject in need thereof comprising administering to the subject an agent comprising an agent that increases an amount of miR-34a in the subject and/or an agent that decreases an amount of miR-129, and an immune checkpoint immunotherapy.

In some embodiments, the present disclosure includes a method of treating or preventing an EBV-related cancer in a subject in need thereof comprising administering to the subject a first agent comprising (a) an agent that increases an amount of miR-34a in the subject and/or (b) an agent that decreases an amount of miR-129, a second agent comprising an immune checkpoint immunotherapy and a third agent that is or comprises at least one other cancer biologic, therapeutic, chemotherapeutic or drug.

In some embodiments, the present disclosure relates methods of treating cancer (e.g. EBV-related cancers) and/or co-formulations including various cancer biologics, therapeutics, chemotherapeutics, or drugs known in the art. For exemplary purposes only, and not intending to be limiting, the following drugs may be used in the present invention: daunorubicin, doxorubicin, epirubicin, idarubicin, adriamycin, vincristine, carmustine, cisplatin, 5-fluorouracil, tamoxifen, prodasone, sandostatine, mitomycin C, foscarnet, paclitaxel, docetaxel, gemcitabine, fludarabine, carboplatin, leucovorin, tamoxifen, goserelin, ketoconazole, leuprolide flutamide, vinblastine, vindesine, vinorelbine, camptothecin, topotecan, irinotecan hydrochloride, etoposide, mitoxantrone, teniposide, amsacrine, merbarone, piroxantrone hydrochloride, methotrexate, 6-mercaptopurine, 6-thioguanine, cytarabine (Ara-C), trimetrexate, acivicin, alanosine, pyrazofurin, pentostatin, 5-azacitidine, 5-azacitidine, 5-Aza-5-Aza-2′-deoxycytidine, adenosine arabinoside (Ara-A), cladribine, ftorafur, UFT (combination of uracil and ftorafur), 5-fluoro-2′-deoxyuridine, 5-fluorouridine, 5′-deoxy-5-fluorouridine, hydroxyurea, dihydrolenchiorambucil, tiazofurin, oxaliplatin, melphalan, thiotepa, busulfan, chlorambucil, plicamycin, dacarbazine, ifosfamide phosphate, cyclophosphamide, pipobroman, 4-ipomeanol, dihydrolenperone, spiromustine, geldenamycin, cytochalasins, depsipeptide, 4′-cyano-3-(4-(e.g., ZOLADEX) and 4′-cyano-3-(4-fluorophenylsulphonyl)-2-hydroxy-3-methyl-3′-(trifluoromethyl)propionanilide.

In one aspect, the disclosure provides a method for treating an EBV-related cancer in a subject in need thereof, comprising administering (i) an effective amount of one or more of (a) an agent that increases an amount of miR-34a in the subject and (b) an agent that decreases an amount of miR-129 in the subject, and (ii) an effective amount of an immune checkpoint immunotherapy selected from an agent that modulates one or more of PD-1, PD-L1, PD-L2, ICOS, ICOSL, and CTLA-4.

In one aspect, the disclosure provides a method for potentiating an immune checkpoint immunotherapy of an EBV-related cancer in a subject in need thereof, comprising administering an agent that increases an amount of miR-34a in the subject, wherein: the immune checkpoint immunotherapy is an agent that modulates one or more of PD-1, PD-L1, and PD-L2 and the subject is predicted to be poorly responsive or non-responsive to the immune checkpoint immunotherapy or has presented as poorly responsive or non-responsive to the immune checkpoint immunotherapy.

In one aspect, the disclosure provides a method for potentiating immune checkpoint immunotherapy of an EBV-related cancer in a subject in need thereof, comprising administering an agent that decreases an amount of miR-129 in the subject, wherein the immune checkpoint immunotherapy is an agent that modulates one or more of ICOS, ICOSL, and CTLA-4.

In one aspect, the present disclosure provides a method of evaluating a subject's cancer, including but not limited to diagnosis, prognosis, and response to treatment. In one aspect, evaluating an EBV-related cancer subject's likelihood of response to an immune checkpoint immunotherapy, comprises evaluating a level of one or more of miR-34a and miR-129 in a biological sample from the subject, wherein a low level of miR-34a and/or high level of miR-129 is indicative of a cancer that is suitable for immune checkpoint immunotherapy.

In some embodiments, treating an EBV-related cancer, comprises: (a) evaluating a subject's likelihood of response to an immune checkpoint immunotherapy, comprising evaluating a level of one or more of miR-34a and miR-129 in a biological sample from the subject, wherein a low level of miR-34a and/or high level of miR-129 is indicative of a cancer that is suitable for immune checkpoint immunotherapy and (b) administering an immune checkpoint immunotherapy selected from an agent that modulates one or more of PD-1, PD-L1, and PD-L2 based on low on expression of PD-1, PD-L1, and PD-L2 to the subject having a low level of miR-34a and/or high level of miR-129.

In some embodiments, a high level of miR-34a indicates a cancer that is evading an anti-cancer immune response through a negative immune signal mediated by one or more of PD-1, PD-L1, and PD-L2. In some embodiments, a low level of miR-129 indicates a cancer that is prevented from delivering a positive anti-tumor signal mediated by one or more of ICOS and ICOSL. In some embodiments, a low level of miR-34a indicates a high likelihood of response to an immune checkpoint immunotherapy selected from an agent that modulates one or more of PD-1, PD-L1, and PD-L2. In some embodiments, the biological sample is a tumor sample is a biopsy selected from a frozen tumor tissue specimen, cultured cells, circulating tumor cells, and a formalin-fixed paraffin-embedded tumor tissue specimen. In some embodiments, the subject is predicted to be poorly responsive or non-responsive to the immune checkpoint immunotherapy based on expression of one or more of PD-1, PD-L1, PD-L2, ICOS, ICOSL, and CTLA-4 in a tumor specimen. In some embodiments, the subject is predicted to be poorly responsive or non-responsive to an agent that modulates one or more of PD-1, PD-L1, and PD-L2 based on low on expression of PD-1, PD-L1, and PD-L2.

### Kits

The invention also provides kits that can simplify the administration of any agent described herein, such as an agent that increases an amount of miR-34a, an agent that decreases an amount of miR-129 and an immune checkpoint immunotherapy. In some embodiments, an immune checkpoint immunotherapy selected from an agent that modulates one or more of PD-1, PD-L1, PD-L2, ICOS, ICOSL, and CTLA-4. An exemplary kit of the invention comprises any composition described herein in unit dosage form. In some embodiments, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can further comprise one or more additional agent, such as a biologic, therapeutic, chemotherapeutic or drug described herein. In some embodiments, the kit comprises a container containing an effective amount of a composition of the invention and an effective amount of another composition, such those described herein.

### Examples

In order that the invention disclosed herein may be more efficiently understood, examples are provided below. It should be understood that these examples are for illustrative purposes only and are not to be construed as limiting the invention in any manner.

### Example 1

### Experimental Methods

Cells: Mutu I and, Mutu III, Daudi, Jijoye, are EBV positive BLs. LCL is an EBV positive cell line. OMA4, DG75 and BL41 are EBV negative BLs. U2932, SUDHL5 are EBV negative GC type DLBCLs. ER/EB 2.5 is an estradiol inducible EBNA2 carrying cell line. The details of the cell lines infected with recombinant EBV and EBNA2/LMP1 transfectants have been described previously.

Infection with a Recombinant EBV Strain

The recombinant strain of Akata EBV was received from Kenzo Takada (Hokkaido University, Sapporo, Japan). The induction of lytic replication, virus production by engaging IgG with corresponding antibodies and infection procedure has been described in detail previously. The supernatant containing recombinant EBV was used to infect EBV negative U2932, SUDHL5, OMA4 and DG75 cells.

### EBNA2 and LMP1 Transfection and Selection

An EBNA2 expression vector J144-C1, the expression vector for LMP1 J132-G5 and the corresponding vector control pSV-MPA GPT were individually transfected into U2932 DLBCL cells by electroporation. The transfection and selection details have been described by us previously. BL41K3 cells transfected with estrogen inducible EBNA2 were treated with 1 μM estradiol to induce EBNA2 expression.

Quantitative RT-PCR (qRT-PCR)

Total RNA from cell lines was isolated using Direct-zol RNA MiniPrep Plus kit (Zymo Research) according to the vending company's instructions. The integrity of RNA was routinely checked using 1% agarose gel and RNA quantification was estimated with a DS-11 spectrophotometer (DeNovix). The cDNA synthesis for mature miR-34a was performed according to the manufacturer's instructions (miScript II RT Kit, Qiagen). For verification of pre-miR-34a expression, reverse transcription qPCR was performed.

### Immunoblotting

EBNA2 and LMP1 expression was verified by monoclonal antibodies PE2 and S12 monoclonal antibodies respectively. B-actin antibodies were purchased from Sigma. PD-L1(E1L3N, cat#13684) and p21 (#2947) and BCL 2 (#15071) were purchased from Cell Signaling.

miR-34a Promoter and Biosensor Luciferase Reporters

To investigate the effect of EBNA2 on miR-34a promoter, 50 thousand EBNA2 expressing U2932 and BL41 cells were seeded in triplicates in a 96 well plate. The cells were co-transfected with 1 μg/μl of pRL-TK luciferase control reporter (Promega) and 0.5 μg/μl miR-34aP luciferase reporter, which carries the wild type miR-34a promoter (Addgene plasmid #50827). After 48 h, the cells were harvested and lysed in 80 μl passive lysis buffer (Promega). Firefly and Renilla luciferase activity was detected by GloMax explorer luminometer (Promega). To verify miR-34a mimic transfection efficiency in U2932 194 and EBNA2 clone 1, 50 nM miR-34a mimics were co-transfected with 20 ng of miR-34a or miR-34a mismatch biosensors. Both biosensors were generated by cloning the reverse miR-34a or miR-34a mismatched complementary sequence cloned downstream of the Renilla promoter in the psiCheck-2 dual luciferase reporter plasmids (Promega). After 48 h post-transfection, the cells were lysed in 80 μl passive lysis buffer (Promega). Firefly and Renilla luciferase activity was detected by GloMax explorer luminometer (Promega).

### PD-L1 Luciferase Reporters and Activity

PD-L1 3′UTR Luciferase reporter construct was made as follows. The full-length PD-L1 3′UTR (2674 bp) (ref|NM_014143.3|Homo sapiens CD274 molecule (CD274), transcript variant 1, mRNA) was PCR amplified from human genomic DNA (Thermo Fisher #4312660), in three separate fragments. Fragment 1 was generated with primers F: GAGACGTAATCCAGCATTGG (SEQ ID NO: 1) and R1: CTGAGGTCTGCTATTTACTGG (SEQ ID NO: 2); Fragment 2 was generated with primers F1: CCAGTAAATAGCAGACCTCAG (SEQ ID NO: 3) and R2: GACTAGATTGACTCAGTGCAC (SEQ ID NO: 4); Fragment 3 was generated with primers F2: GTGCACTGAGTCAATCTAGTC (SEQ ID NO: 5) and R: TAACTTTCTCCACTGGGATG (SEQ ID NO: 6). The three fragments were connected by overlap PCR, with forward primer:

(SEQ ID NO: 7)
actcgagGAGACGTAATCCAGCATTGG (containing a XhoI
site, underlined)
and
reverse primer:
(SEQ ID NO: 8)
agcggccgcTAACTTTCTCCACTGGGATG (containing a NotI
site, underlined).

The full-length PD-L1 3′UTR was cloned into the Psicheck2 vector between the XhoI and NotI sites downstream of Renilla luciferase, and fully verified by sequencing. Site-directed mutagenesis of PD-L1 3′UTR

Point mutations were introduced into the miR-34a seed sequence of 3′UTR of PD-L1 cloned in Psicheck-2 vector according to the QuikChange site-directed mutagenesis kit (Agilent Technologies). The mutagenic primers containing the desired mutation in the miR-34a seed sequence of the 3′UTR of PD-L1 were: Forward primer: 5′-221 3′ and the reverse primer: 5′-CATATGAATGAACGTTCGTAGCAGTTGCTTC-3′ (SEQ ID NO: 9). The miR-34a seed sequence in the wild type 3′-UTR of PD-L1 is in bold letters: 5′GAAGCAACTGCTACTGCCTTTCATTCATATG-3′ (SEQ ID NO: 10). TGCCT was mutated to GAACG. The mutated seed sequence was verified by sequencing.

### EBF1 Knockdown

Knock-down of EBF1 was obtained by transduction of U2932 and its EBNA2 expressors with pLK0.1 lentiviral vectors, which carry shEBF1 and the corresponding control shRNA (TRC Human EBF1 shRNA, Clone ID: TRCN0000013831 and Plko.1-emptyT control TRCN0000208001, Open Biosystems, Dharmacon). Cells were transduced as described below and were selected with 1.5 μg/ml puromycin for 10 days and used for further experiments.

### Lentivirus Transduction

The cell lines U2932 MPA vector and U2932 EBNA2 were transduced with pLL3.7 hsa-miR-34a, (Addgene plasmid #25791) and pLL3.7 control vector (Addgene plasmid #11795). For the production of lentiviruses, viable and confluent HEK293T cells were transfected (Fugene6, Promega) with the transfection mixture composed of 10 μg of pLL3.7 hsa-miR-34a or pLL3.7 vector control along with 5 μg pMD2.G envelope plasmid (Addgene plasmid #12259) and 5 μg psPAX2 packaging plasmid (Addgene plasmid #12260).

### Standard Mixed Lymphocyte Reaction (MLR)

PBMCs were isolated from the blood of healthy donors using Ficoll-Paque separation media (GE Healthcare) and were seeded in 24-well non-tissue culture-treated plates (Falcon, Fisher, Pittsburgh, Pa., USA), previously coated with anti-CD3 (clone-UCHT1; Pharmingen, San Diego, Calif., USA) and anti-CD28 (clone-CD28.2; Pharmingen, San Diego, Calif., USA) at the concentration of 1 μg/mL in phosphate-buffered saline (PBS) at 0.4 mL/well overnight at 4° C. The day after, the plates were washed in 1×PBS and PBMCs were added to the CD3/CD28 coated 250 wells at a density of 1×10⁶ cells/well and cultured for 72 h, in order to activate the CD4 and CD8 cell population. One day before seeding the stimulators, 1×10⁵ U2932 MPA vector and U2932 EBNA2 CL-1 were transiently transfected with 50 nM mimic negative control and mimic miR-34a (Ambion) and subsequently irradiated with a sub-lethal dose of 5Gy for 2 minutes. The cells were placed in co255 culture with 1×10⁶ PBMCs. At 72 h post-transfection and after 48 h co-culture, all samples were treated for 5 h with GolgiStop™ (BD biosciences) to block cytokine accumulation in the Golgi complex, for the detection of IFN-γ producing cells, by flow cytometry. The entire population of co-cultured cells were stained with FITC mouse anti-human CD8 and Pacific Blue mouse anti-human CD4 (BD Pharmingen) for detection of T cells. The same cells were then permeabilized with cytofix/cytoperm buffer (BD Pharmingen), according to the manufacturer's instructions. The cells were stained intracellularly with human IFN-γ, R-PE, (Invitrogen). A matched isotype control, anti-Human IgG Fc secondary antibody, PE (Invitrogen), was also included in this experiment. Sample acquisition was performed with Gallios Flow Cytometer. The data were analyzed with Kaluza for Gallios Software.

3D microfluidic platform for T cell responses to EBNA2 transfected U2932 DLBCL. The 3D microfluidic chips, polydimethylsiloxane (PDMS, Sylgard 184, Dow-Corning, Midland, Mich.) microfluidic devices were fabricated using soft lithography as described previously. The devices were treated with 0.01% v/v poly-L-lysine and 0.5% v/v gluteraldehyde to promote collagen/fibronectin adhesion. After washing over-night in water, steel acupuncture needles (160 μm diameter, Seirin, Kyoto, Japan) were introduced into the devices and a solution of 2.5 mg/ml type 1 collagen, 1× M199 medium, 1 mM HEPES, 0.1 M NaOH and NaHCO₃(0.035% w/v) and 200 ng/ml Fibronectin (Thermo Fisher Scientific, Waltham, Mass.), was infused into the devices and allowed to polymerize for 40 min at 37° C. Subsequently, needles were removed to create 160 μm diameter channels within collagen/275 fibronectin gel and cells were introduced into devices. In co-culture experiments, each device was first seeded with 5×10³ U2932 EBNA2 CL-1 transduced with the control lentivirus or miR-34a containing lentiviral vector and were incubated for 24 hrs at 37° C. Subsequently, 5×10⁴ PBMCs, containing previously activated T cells were added in complete medium (RPM1 1640/10% FBS). The devices were in triplicates and incubated for an additional hrs before performing immunostaining. For immunostaining of the co-cultures in microfluidic devices, the cells were fixed with 4% PFA for 10 minutes and washed twice in PBS, permeabilized with 0.1% (v/v) Triton X 100 in PBS for 20 minutes at room temperature, and treated with a blocking solution (BSA 5% in PBS 0.1% Triton X 100). The devices were incubated with rabbit anti-caspase 3 (Cell Signaling) or mouse anti-CD4 and -CD8 antibodies (1:100 dilutions, Biolegend) and kept on a rocking platform 0/N at 4° C. Devices were merged in PBS and left on a rotor 0/N, at 4° C. to remove excess antibody. The day after, PBS was removed and Alexa 568-conjugated goat-anti-rabbit IgG (primary abs, caspase 3) and Alexa 647-conjugated goat-anti-mouse IgG (primary abs, CD4 and CD8) secondary antibodies diluted 1:100 in blocking buffer, were added per well in each device 0/N at 4° C. Finally, PBS was added in each device and processed to detect caspase-3, CD4 and CD8 staining. The devices were visualized using confocal microscope (LSM 710, Carl Zeiss), and image analysis made by Image J by performing a maximum intensity z projection and merging the channels.

PD-L1 Immunohistochemistry and Quantitative Analysis in Biopsies from DLBCL Patients

A written informed consent was obtained from all patients involved in the study. The study design was approved by the Institute ethics review board. Paraffin sections were immunostained for PDL-1, PD-1, EBNA2, LMP1, MUM-1, CD10 and Bcl-6, using an automated immunostainer (DAKO, Glostrup, Denmark). As control for PD-L1 immunostaining, sections from paraffin embedded human lung carcinoma were used. For quantitative IHC analysis, the Aperio Imagescope algorithm was used to evaluate both percentage positive cells and intensity the stained tumor cells in three regions of three clinical samples representing each of the three types non-GC DLBCL category (EBV neg, EBV+/EBNA− and EBV+/EBNA2+).

### Example 2

### PD-L1 Expression is Induced in Latency III Expressing BLs, In Vitro Infected BLs and DLBCLs and EBNA2 Transfected Cells

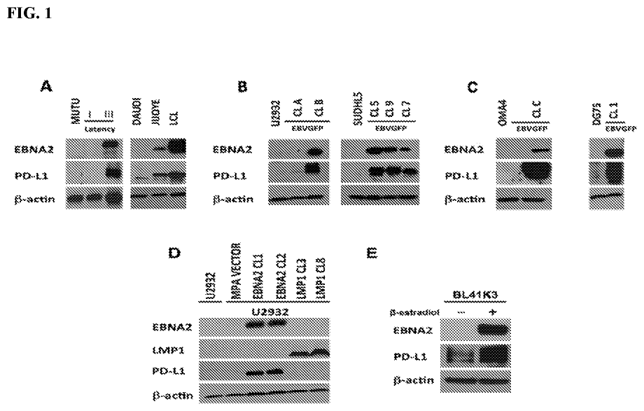

The restricted latency expressor cell line Mutu I (53) did not express PD-L1 while its EBNA2 expressing counterpart showed increased PD-L1. Two additional BL cell lines, Jijoye, which is positive for EBNA2 expression expressed PD-L1, while EBNA2 deleted Daudi BL lacked PD-L1 expression (FIG. 1A). The above data suggest that latency III related viral proteins could influence PD-L1.

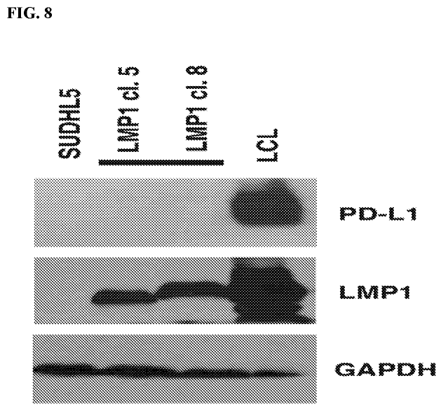

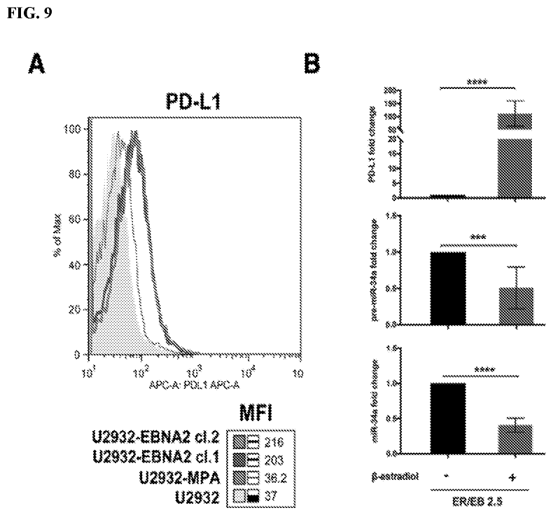

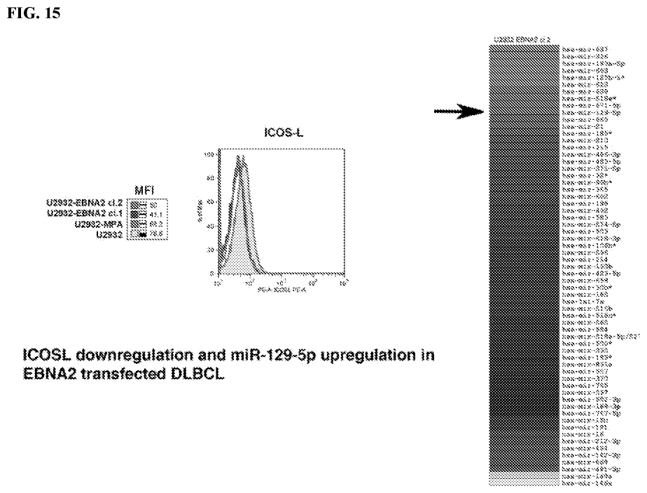

To extend these observations, two EBV negative GC DLBCLs, U2932 and SUDHL5 and two EBV negative BLs, OMA4 and DG75 were infected with a recombinant Akata EBV. The resultant convertants expressed EBNA2 (FIGS. 1B and 1C). PD-L1 expression was strongly upregulated in both DLBCLs (FIG. 1B) and two BLs (FIG. 1C) after in vitro EBV infection. From data in FIG. 1A (right panel, Daudi-Jijoye comparison), it became clear that EBNA2 might have a critical role in the observed upregulation of PD-L1. To further confirm this, U2932 DLBCL was transfected with an EBNA2 containing expression vector. The transfection and selection conditions of EBNA2 and LMP1 expressing derivatives of U2932 have been previously described. A strong increase in PD-L1 was observed in EBNA2 transfectants but not in LMP1 transfected U2932 cells (FIG. 1D, left panel). The lack of PD-L1 induction by LMP1 was also confirmed in transfected SUDHL5 DLBCL (FIG. 8). PD-L1 induction by EBNA2 was also confirmed by flow cytometry as well in EBNA2 expressing U2932 (FIG. 9A). Additionally, in BL41 K3 cells, EBNA2 induction by estradiol treatment was paralleled by an increase in PD-L1 expression (FIG. 1E). PD-L1 upregulation was confirmed by real time q-PCR in ER/EB 2.5 cell line, which carries estradiol inducible EBNA2 (FIG. 9B). Similarly, as shown in FIG. 15, detection of ICOSL by flow cytometry in U2932 and its EBNA2 expressing derivatives showed a decrease in ICOSL and an increase of miR-129-5p in EBNA2 transfected DLBCL, confirmed in the microarray (FIG. 15 left panel).

Transcription of the PD-L1 Targeting miRNA miR-34a is Downregulated by EBNA2

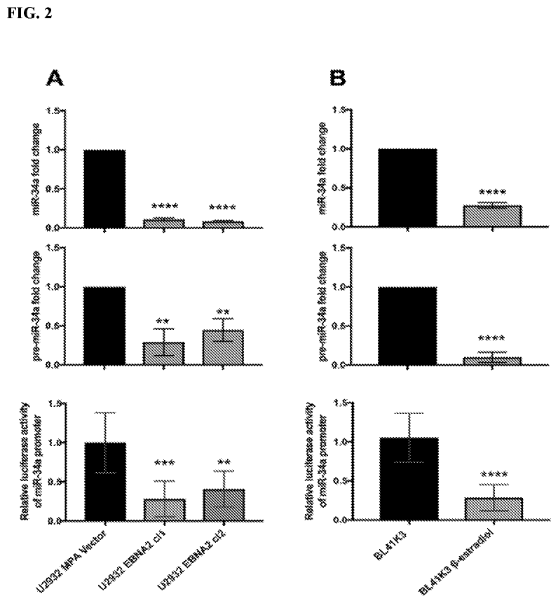

Previous data has shown that EBNA2 can profoundly alter cellular miRNA expression profile in U2932 cells. Given the strong increase of PD-L1 expression in EBNA2 transfected BL and DLBCLs and since miR-34a targets PD-L1, it was examined if miR-34a expression is affected in EBNA2 expressing B lymphoma cells. As shown in FIG. 2A, top panel, EBNA2 transfected U2932 cells showed a marked decrease in miR-34a. Similarly, BL41K3 carrying estrogen inducible EBNA2 showed reduced miR-34a after estrogen treatment (FIG. 2B, top panel). Additionally, both U2932 EBNA2 and BL41K3 cells showed reduced pre-miR-34a expression, (FIG. 2A and FIG. 2B, middle panels). To further confirm that the miR-34a decrease is transcriptional, EBNA2 expressing U2932 and BL41 cells were transfected with miR-34a promoter carrying Luc reporters. As seen in FIG. 2A and FIG. 2B (lower panels), in the presence of EBNA2, the luciferase activity was significantly reduced, confirming that miR-34a is indeed transcriptionally affected by EBNA2.

Validation of the PD-L1 3′UTR as a miR-34a Target in U2932 DLBCL

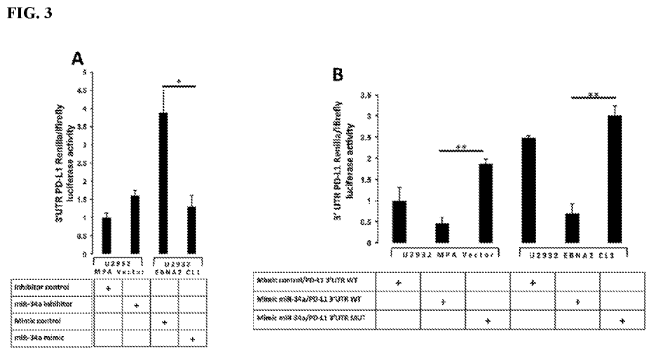

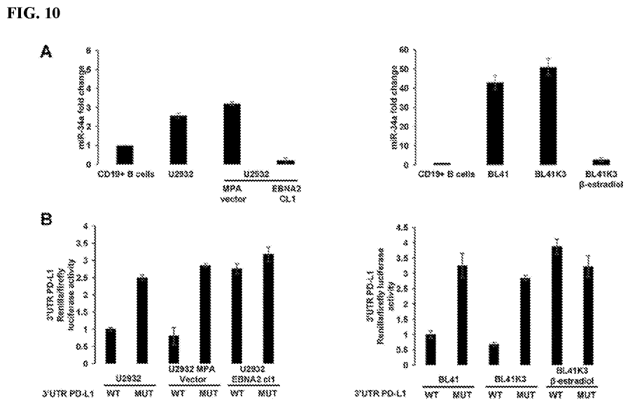

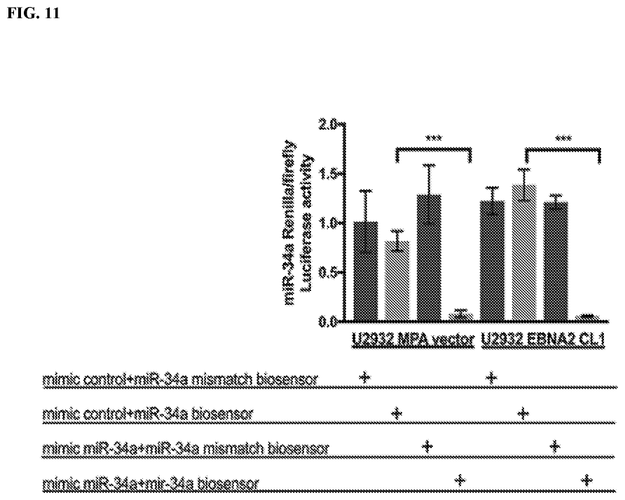

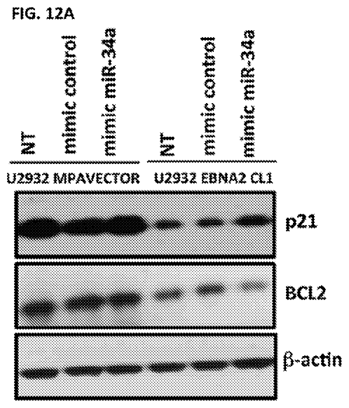

To investigate the role of miR-34a in the regulation of PD-L1 3′ UTR, the complete 3′UTR of PD344 L1 was cloned into a luciferase reporter construct and transfected into U2932 MPA vector and U2932 EBNA2 cells. Subsequently, the miR-34a inhibitors were introduced into the vector alone carrying cells where miR-34a was higher. Instead, miR-34a mimics were transfected into EBNA2 expressing counterparts with low miR-34 expression. FIG. 3A shows luciferase activity in control and in presence of miR-34a inhibitor in U2932 MPA vector or miR-34a mimic in the EBNA2 transfectant. In accordance with miR-34a downregulation in U2932 EBNA2, the luciferase activity was high in these cells. When mimic miR-34a was introduced into EBNA2 expressing cells, the reporter gene activity was significantly reduced (FIG. 3A). To confirm the specificity of miR-34a binding in PD-L1 3′UTR, miR-34a seed sequence was mutated using site-directed mutagenesis. As seen in FIG. 3B, the wild type 3′ UTR reporter activity was high, consistent with low miR-34a in EBNA2 expressing cl-1. When miR-34a mimic was introduced into these cells, the luciferase activity was reduced. In contrast, the mutated seed sequence carrying luciferase reporters were no longer repressed by miR-34a. This not only validated 356 the sequence specificity of the miRNA-mRNA binding but also mapped and confirmed the miR-34a recognition sequence in the PD-L1 3′UTR. The absolute expression of miR-34a in U2932 and BL41 parental cell lines and their EBNA2 expressing counterparts in comparison with CD19+ B cells and the Luc activity of wild type and mutated 3′PD-L1 UTR in both cell lines is shown in FIG. 11. In comparison with normal CD19+ B cells, both U2932 and BL41 had higher levels of miR-34a (FIG. 10A). As a consequence, the luciferase activity of the wild type 3′ PD-L1 UTR construct was repressed, which indicates miR-34a binding to the 3′UTR of PD-L1. In contrast, luciferase activity of the mutated 3′ PD-L1 UTR was not affected by miR-34a. Similarly, in EBNA2 transfected cells, due to lower expression of miR-34a, the Luc activity from both WT and mutated 3′UTR construct was not affected. (FIG. 10B).

Over-Expression of miR-34a in U2932 EBNA2 Cells Reduces PD-L1

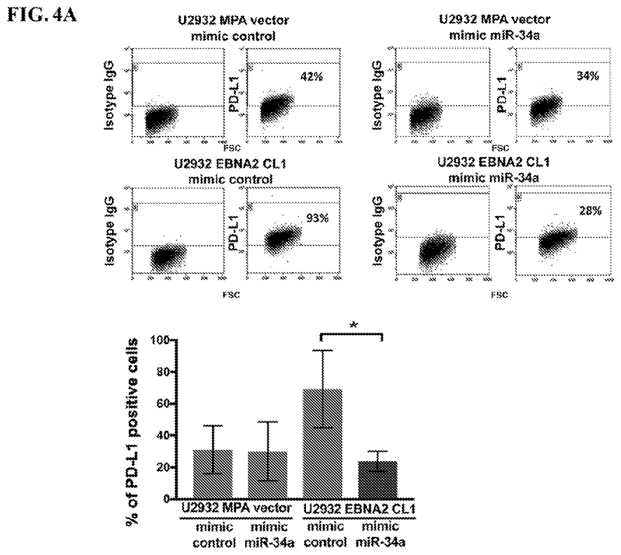

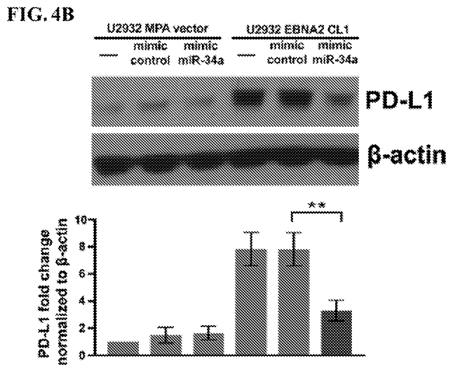

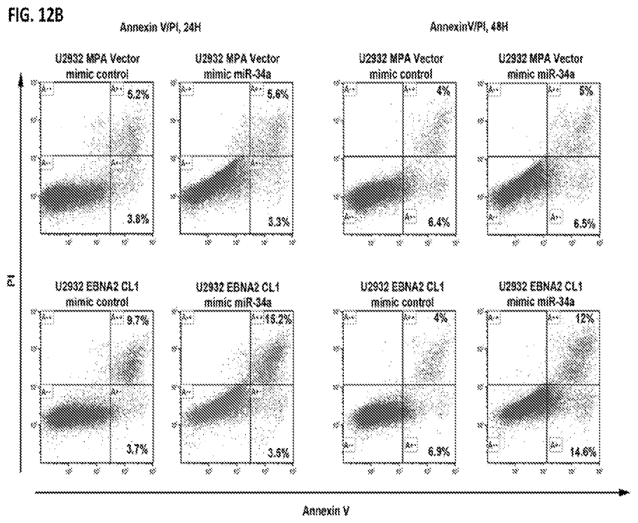

Having established that miR-34a binds to 3′UTR of PD-L1, it was next examined if miR-34a over expression could have a direct effect on PD-L1. For this purpose, we transfected miR-34a mimics in U2932 EBNA2 CL-1. As seen in FIG. 11, the decrease in Luc activity of the biosensor psicheck-2 construct in the presence of miR-34a mimic clearly suggests its successful delivery and binding to target sequences. To investigate the direct effect of miR-34a on PD-L1, miR-34a transfected U2932 EBNA2 CL-1 was analyzed for PD-L1. A significant reduction in PD-L1 was observed after over-expression of miR-34a in comparison to the scrambled control (FIG. 4A and FIG. 4B). It was further investigated if over-expression of miR-34a in U2932 cells influences p21 and BCL2, previously shown to be regulated by this miRNA. As shown in FIG. 12A, U2932 EBNA2 CL-1 transfected with miR-34a had an increased p21 but reduced BCL2. Consequently, the number of apoptotic cells was higher in miR-34a transfected U2932 EBNA2 CL-1 in comparison with the vector transfected cells (FIG. 12B).

EBF1 Knockdown De-Represses miR-34a and Downregulates PD-L1 in U2932 EBNA2 Cells

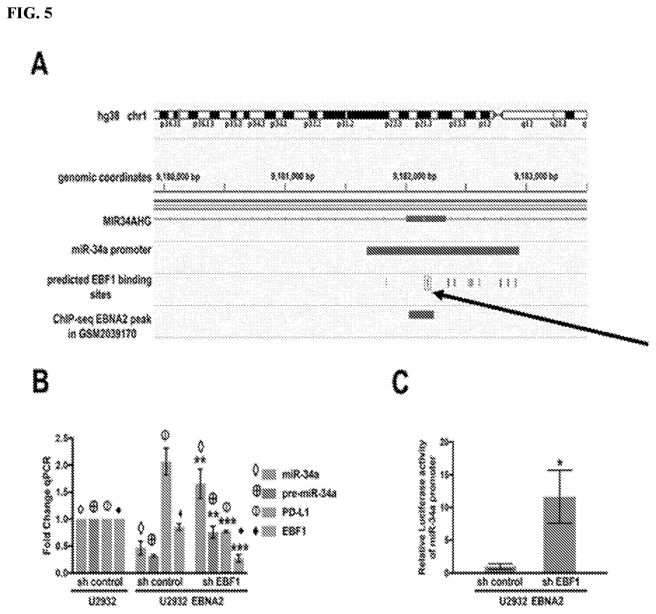

Previously reported ChIP-Seq data 384 show that EBNA2 co-localizes with EBF1 at promoter/enhancers of many genes. To identify the molecular mechanism of miR-34a regulation by EBNA2, EBNA2 ChIP-Seq datasets from GEO database (accession number: GSM2039170) were analyzed and it was found that EBNA2 peaks at the miR-34a promoter. Subsequently, through JASPAR database and visualization through Integrative Genomics Viewer (IGV), using the reference hg38 (human genome), results found multiple predicted binding sites for EBF1 at the miR-34a promoter, and among them, one consensus EBF1 sequence overlaps with the EBNA2 peak (FIG. 5A, see arrow pointing square in FIG. 5A). Based on this, it was reasoned that miR-34a might be regulated by EBNA2 through EBF1. To verify this, the parental U2932 and its EBNA2 expressing derivative line were transduced with lentiviral vectors carrying shEBF1 and sh-control. As shown in FIG. 5B, upon EBF1 knockdown in U2932 EBNA2 cl-1, miR-34a and pre-miR-34a expression is depressed with a consequential decrease in PD-L1. It was further found that miR-34a promoter activity was increased upon EBF1 K.D. (FIG. 5C). These data establish a circuit where EBNA2 might recruit EBF1 to miR-34a promoter to downregulate its expression and consequently upregulate PD-L1.

Suppression of T Cell Activation by EBNA2 and Increased Immunogenicity after miR-34a Over Expression as Measured in MLR and 3D Biomimetic Microfluidic Platforms

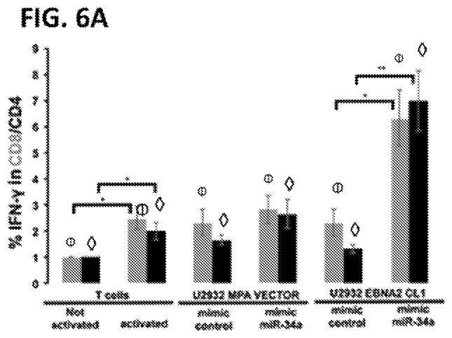

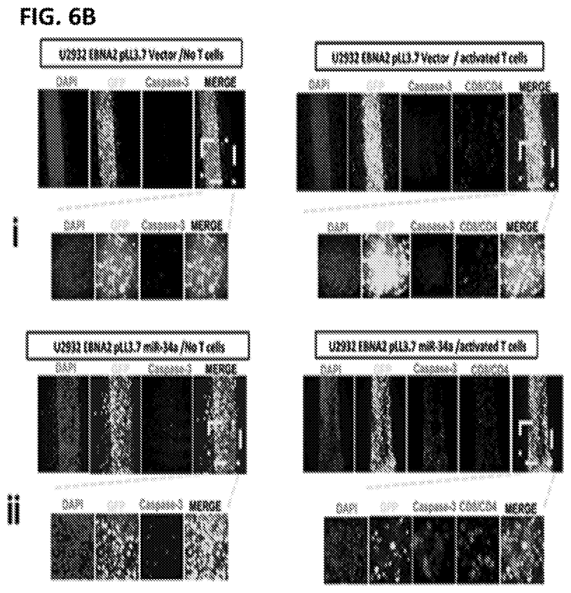

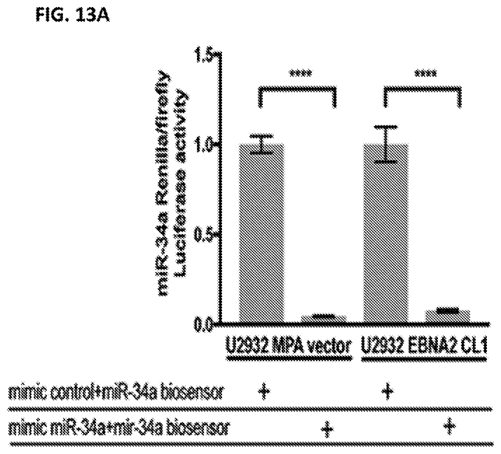

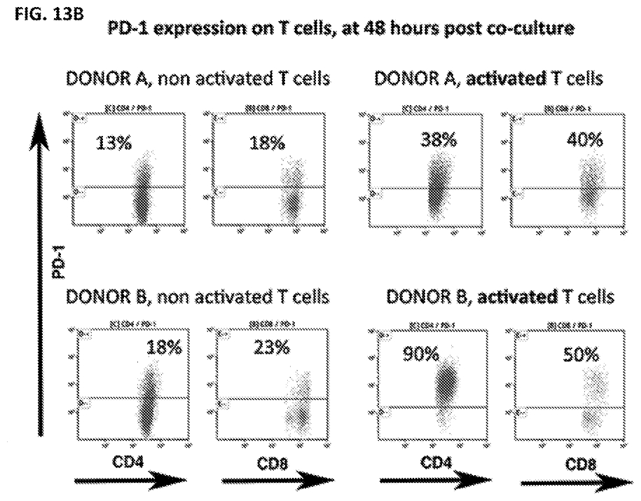

In order to understand the immunological relevance of PD-L1 upregulation and miR-34a downregulation by EBNA2, a MLR assay was first employed. After three days of PBMC activation on CD3/CD28 coated wells, the irradiated stimulator U2932 MPA vector, U2932 EBNA2 CL-1 and either their mimic control or miR-34a transfected derivatives were added in a MLR. Successful miR-34a delivery in stimulator cells and its binding to specific target sequence was confirmed using the psicheck-2 biosensor reporter assay (FIG. 13A). Effector T cell activation was confirmed by a strong increase in PD-1 expression in two donors (FIG. 13B). The activated T cell state was corroborated by increased IFN-γ production (FIG. 6A). Importantly, U2932 EBNA2 CL-1 boosted IFN-γ production, by both CD8 and CD4 T cells, only when miR-34a was over-expressed (FIG. 6A). These data suggest that the increase in PD-L1 by EBNA2 may have a negative effect on T cell activation and reconstitution of miR-34a restores immunogenicity of EBNA2 transfectants.

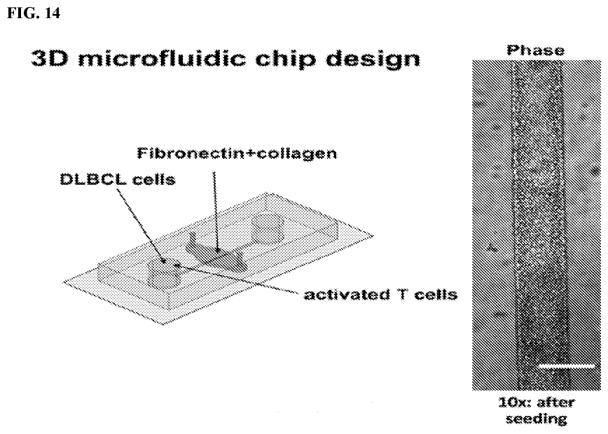

How miR-34a might reverse the poor immunogenicity of EBNA2-transfected high PD-L1 expressing U2932 cells was next examined. The schematic design of the 3D microfluidic chip based coculture system is shown in FIG. 14. The effector T cell activation was confirmed by increased IFN-γ and stimulator U2932 EBNA2 cells transduced either with lentiviral vectors carrying miR-34a or vector control, were introduced into microfluidic devices. The expression of miR-34a in lentivirus transduced U2932 EBNA2 cells was checked by real time qPCR and the consequent PD-L1 decrease was verified by flow cytometry. FIG. 6B (panel (i)) shows the device with empty lentiviral vector transduced U2932 EBNA2 expressors either in the presence or absence of T cells. No significant change in caspase-3 expression was observed. In contrast, as seen in FIG. 6B (panel (ii)), when miR-34a containing lentivirus was transduced into EBNA2 U2932 clone, there was a marginal induction of caspase-3 in the absence of T cells, most probably due to apoptosis induced by miR-34a expression. In contrast, over-expression of miR-34a in EBNA2 expressing U2932, in the presence of CD4/CD8 cells, induced significant tumor cell death, as indicated by increased caspase-3 expression (FIG. 6B (panel (ii) and FIG. 6C)). Overall, these data suggest that reconstitution of miR-34a in EBNA2 expressing U2932 makes them more immunogenic.

### Example 3: PD-1,1 and EBV Correlation in Clinical DLBCL Samples

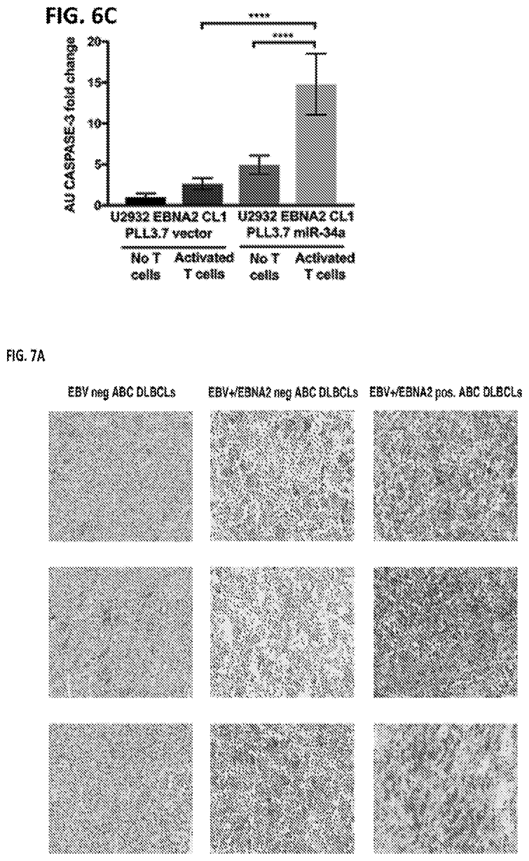

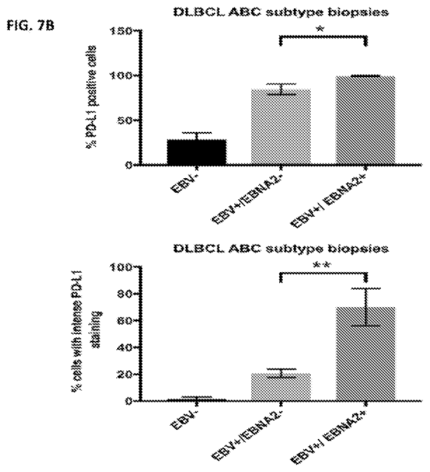

In a cohort of 27 cases of DLBCLs, how EBV and EBNA2 expression is correlated with increase in PD-L1 expression was investigated. According to the Hans Algorithm, 21 cases were classified as non-GC type and 6 cases as GC type. FIG. 7A shows PD-L1 expression in three non-GC DLBCLs representing each category namely, EBV negative, EBV+/EBNA2- and EBV+/EBNA2+ samples. PDL-1 expression was detected at the cell membrane level, in the cytoplasm or as dots in the Golgi area of the neoplastic cells. For quantitative estimation of PD-L1 expression and staining intensity, Aperio Imagescope analysis was employed. The stained tissue sections were digitalized at a 40× magnification using Aperio Scan Scope. The percentage positivity was calculated by counting positive cells in three squared areas measuring 50000 μm2 from each clinical sample. In the same areas the number of the positive cells was determined using the Aperio software IHC Membrane v1. The IHC Membrane Image Analysis algorithm detects membrane staining for individual tumor cells in the selected regions and quantifies the intensity and completeness of the membrane staining. FIG. 7B upper panel shows that there was a slight and statistically significant overall increase in PD-L1 positive cells in EBNA2 positive cases. Notably, as shown in FIG. 7B, in all EBNA2+ samples analyzed, the number of cells with high staining intensity (+2, +3) as measured by Imagescope algorithm, was significantly higher in EBNA2+ ABC DLBCLs in comparison with EBNA2− cases (Table 1). PD-1 expression was also analyzed and found that it is generally expressed by infiltrating cells like T lymphocytes (TILs) and macrophages and not by the neoplastic cells. There was no correlation between the number of PD-1 positive infiltrating cells and PDL-1 expression by neoplastic cells. Table 1 describes the details of the clinical samples.

TABLE 1

| | | | | | % | % intensely |
|---|---|---|---|---|---|---|
| | | | | | PD-L1 | stained cells |
| | | | | PD-L1 | positive | for PD-L1 |
| DLBCL ® | EBV | EBNA2 | Type | staining | cells* | (2+,3+)* |
| 5 | + | EBNA2+ | Non-GC | 5/5 | 100§ | 86§ |
| | | Latency III | | (100%) | 99 | 60 |
| | | | | | 99 | 64 |
| 6 | + | EBNA2− | Non-GC | 6/6 | 87 | 18 |
| | | Latency III | | (100%) | 88 | 24 |
| | | | | | 78 | 20 |
| 10 | − | EBV− | Non-GC | 8/10 | 35 | 1 |
| | | | | (80%) | 30 | 3 |
| | | | | | 19 | 0 |
| 6 | − | EBV− | GC | 3/6 | − | − |
| | | | | PDL-1+ | | |
| | | | | (50%) | | |

A total of 27 DLBCLs (6 GC and 21 non-GC) were included in the study. The classification was done according to Hans Algorithm. In three clinical samples representing each DLBCL category, namely EBV neg, EBV+/EBNA2− and EBV+/EBNA2+ ABC DLBCL, the PD-L1 stained sections were digitalized at a 40× magnification using Aperio Scan Scope. The percentage positivity was calculated by counting positive cells in three squared areas measuring 50000 μm² in three different samples of each category. On average about 500 cells per region were counted. In the same areas the number of positive cells was determined using the Aperio software IHC Membrane v1. The IHC Membrane Image Analysis algorithm detects the membrane staining for the individual tumor cells in the selected regions and quantifies the intensity and completeness of the membrane staining. +1 intensity is partial membrane staining, +2 is moderate and complete staining and +3 is intense and complete membrane staining.

### Example 4: Tumor Immunogenicity is Enhanced by Combining miR-34a and Anti-PD-L1

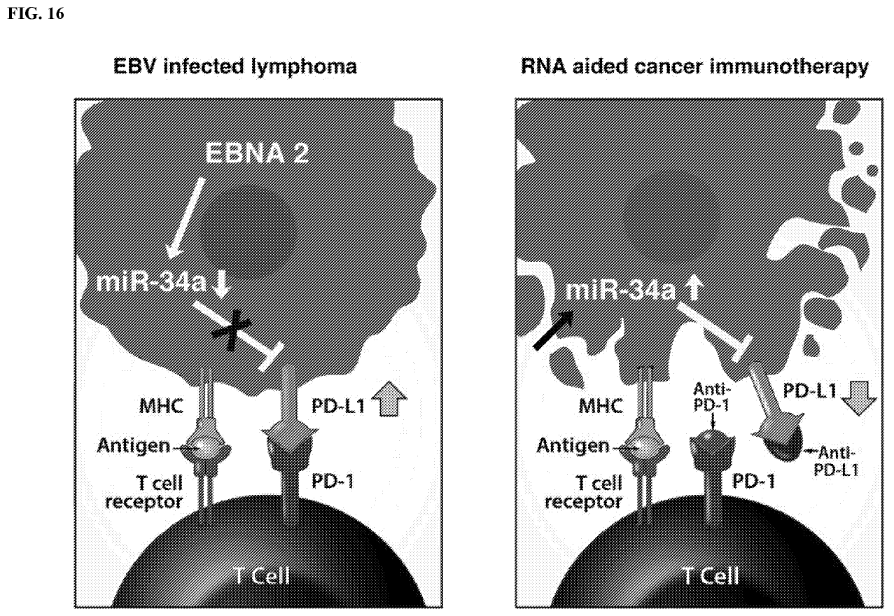

In the experiments of this example, it was hypothesized that tumor immunogenicity is enhanced by combining miR-34a and anti-PD-L1. FIG. 16 shows an image of how RNA aided immunotherapeutics (i.e., a combination of both a miRNA and an anti-PD-L1 antibody) can be used to treat EBV associated cancer.

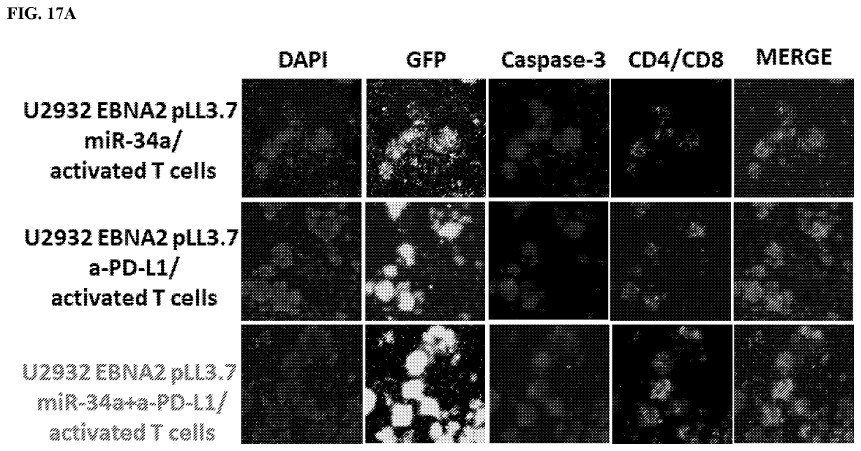

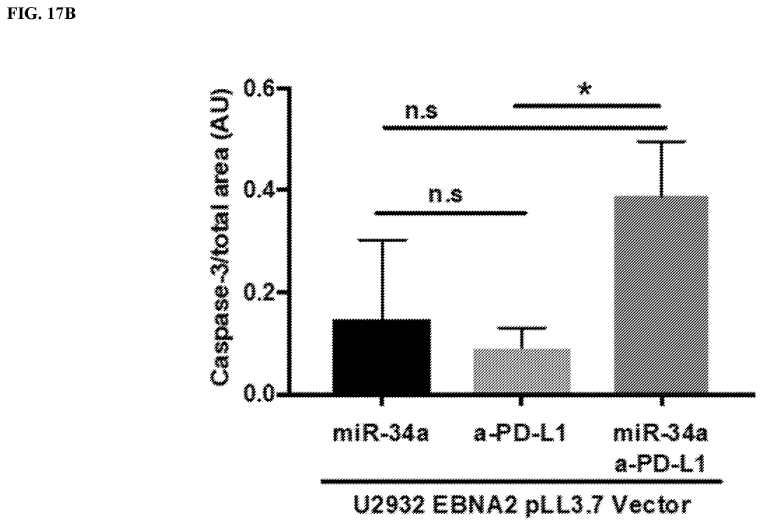

FIG. 17A shows confocal microscope images with U2932 EBNA2 cells transduced with: (1) either a miR-34a or an anti-PD-L1 containing pLL3.7 lentivirus and cocultivated with activated T cells; (2) both a miR-34a and an anti-PD-L1 containing pLL3.7 lentivirus and cocultivated with activated T cells; or (3) the corresponding vector control carrying the GFP marker, and cocultivated with activated T cells. No significant change in caspase-3 expression was observed. However, as seen in the panel on the far right of FIG. 17B, when miR-34a and anti-PD-L1 containing lentivirus was transduced into the EBNA2 U2932 cells, there was an enhanced induction of apoptosis and tumor cell death (merged images, pink, fifth panel, and see FIG. 17B). The combined therapy was synergistic.

Overall, these data suggest that the combination of miR-34a and anti-PD-L1 in EBNA2 expressing U2932 enhances the tumor immunogenicity of these cells.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any way.

### REFERENCES

1. Kuppers R. The biology of Hodgkin's lymphoma. Nature reviews Cancer. 2009; 9(1):15-27.
2. Kuze T, Nakamura N, Hashimoto Y, Sasaki Y, Abe M. The characteristics of Epstein-Barr 575 virus (EBV)-positive diffuse large B-cell lymphoma: comparison between EBV(+) and EBV(−) cases in Japanese population. Japanese journal of cancer research: Gann. 2000; 91(12):1233-40.
3. Young L S, Yap L F, Murray P G. Epstein-Barr virus: more than 50 years old and still providing surprises. Nature reviews Cancer. 2016; 16(12):789-802.
4. Kang M S, Kieff E. Epstein-Barr virus latent genes. Experimental & molecular medicine. 2015; 47: e131.
5. Babcock G J, Hochberg D, Thorley-Lawson A D. The expression pattern of Epstein-Barr virus latent genes in vivo is dependent upon the differentiation stage of the infected B cell. Immunity. 2000; 13(4):497-506.
6. Rowe M, Kelly G L, Bell A I, Rickinson A B. Burkitt's lymphoma: The Rosetta Stone deciphering Epstein-Barr virus biology. Seminars in cancer biology. 2009; 19(6):377-88.
7. Klein G. Tumor Associations of EBV—Historical Perspectives. Current topics in microbiology and immunology. 2015; 390(Pt 1):17-22.
8. Farina A, Farina G A. Fresh Insights into Disease Etiology and the Role of Microbial

Pathogens. Current rheumatology reports. 2016; 18(1):1.
9. Rabson M, Gradoville L, Heston L, Miller G. Non-immortalizing P3J-HR-1 Epstein-Barr virus: a deletion mutant of its transforming parent, Jijoye. Journal of virology. 1982; 44(3):834 44.
10. Nikitin P A, Yan C M, Forte E, Bocedi A, Tourigny J P, White R E, et al. An ATM/Chk2-mediated DNA damage-responsive signaling pathway suppresses Epstein-Barr virus transformation of primary human B cells. Cell host & microbe. 2010; 8(6):510-22.
11. Wang F, Kikutani H, Tsang S F, Kishimoto T, Kieff E. Epstein-Barr virus nuclear protein 2 transactivates a cis-acting CD23 DNA element. Journal of virology. 1991; 65(8):4101-6.
12. Kaiser C, Laux G, Eick D, Jochner N, Bornkamm G W, Kempkes B. The proto-oncogene c myc is a direct target gene of Epstein-Barr virus nuclear antigen 2. Journal of virology. 1999; 73(5):4481-4.
13. Jochner N, Eick D, Zimber-Strobl U, Pawlita M, Bornkamm G W, Kempkes B. Epstein-Barr virus nuclear antigen 2 is a transcriptional suppressor of the immunoglobulin mu gene: implications for the expression of the translocated c-myc gene in Burkitt's lymphoma cells. The EMBO journal. 1996; 15(2):375-82.
14. Boccellato F, Anastasiadou E, Rosato P, Kempkes B, Frati L, Faggioni A, et al. EBNA2 interferes with the germinal center phenotype by downregulating BCL6 and TCL1 in non-Hodgkin's lymphoma cells. Journal of virology. 2007; 81(5):2274-82.
15. Hayward S D. Viral interactions with the Notch pathway. Seminars in cancer biology. 2004; 14(5):387-96.
16. Lu F, Chen H S, Kossenkov A V, DeWispeleare K, Won K J, Lieberman P M. EBNA2 Drives

Formation of New Chromosome Binding Sites and Target Genes for B-Cell Master Regulatory Transcription Factors RBP-jkappa and EBF1. PLoS pathogens. 2016; 12(1):e1005339.
17. Henkel T, Ling P D, Hayward S D, Peterson M G. Mediation of Epstein-Barr virus EBNA2 transactivation by recombination signal-binding protein J kappa. Science (New York, N.Y.).1994; 265(5168): 92-5.
18. Zhao B, Zou J, Wang H, Johannsen E, Peng C W, Quackenbush J, et al. Epstein-Barr virus exploits intrinsic B-lymphocyte transcription programs to achieve immortal cell growth. Proceedings of the National Academy of Sciences of the United States of America. 2011; 108(36): 14902-7.
19. Glaser L V, Rieger 621 S, Thumann S, Beer S, Kuklik-Roos C, Martin D E, et al. EBF1 binds to EBNA2 and promotes the assembly of EBNA2 chromatin complexes in B cells. PLoS pathogens. 2017; 13(10):e1006664.20. Sharma P, Allison J P. Immune checkpoint targeting in cancer therapy: toward combination strategies with curative potential. Cell. 2015; 161(2):205-14.
21. Sharma P, Allison J P. The future of immune checkpoint therapy. Science (New York, N.Y.). 2015; 348(6230):56-61.
22. Anastasiadou E, Jacob L S, Slack F J. Non-coding RNA networks in cancer. Nature reviews Cancer. 2018; 18(1):5-18.
23. Kasinski A L, Slack F J. Epigenetics and genetics. MicroRNAs en route to the clinic: progress in validating and targeting microRNAs for cancer therapy. Nature reviews Cancer. 2011; 11(12):849-64.
24. Raver-Shapira N, Marciano E, Meiri E, Spector Y, Rosenfeld N, Moskovits N, et al. Transcriptional activation of miR-34a contributes to p53-mediated apoptosis. Molecular cell. 2007; 26(5):731-43.
25. Adams B D, Anastasiadou E, Esteller M, He L, Slack F J. The Inescapable Influence of Noncoding RNAs in Cancer. Cancer research. 2015; 75(24):5206-10.
26. Hermeking H. The miR-34 family in cancer and apoptosis. Cell death and differentiation. 2010; 17(2):193-9.
27. Wang L Q, Kwong Y L, Wong K F, Kho C S, Jin D Y, Tse E, et al. Epigenetic inactivation of mir-34b/c in addition to mir-34a and DAPK1 in chronic lymphocytic leukemia. Journal of translational medicine. 2014; 12:52.
28. Merkel 0, Asslaber D, Pinon J D, Egle A, Greil R. Interdependent regulation of p53 and miR-34a in chronic lymphocytic leukemia. Cell cycle (Georgetown, Ill.). 2010; 9(14):2764-8.
29. Wang X, Li J, Dong K, Lin F, Long M, Ouyang Y, et al. Tumor suppressor miR-34a targets P D-L1 and functions as a potential immunotherapeutic target in acute myeloid leukemia. Cellular signalling. 2015; 27(3):443-52.
30. Alizadeh A A, Eisen M B, Davis R E, Ma C, Lossos I S, Rosenwald A, et al. Distinct types of diffuse large B-cell lymphoma identified by gene expression profiling. Nature. 2000; 403(6769):503-11.
31. Kiyasu J, Miyoshi H, Hirata A, Arakawa F, Ichikawa A, Niino D, et al. Expression of programmed cell death ligand 1 is associated with poor overall survival in patients with diffuse large B-cell lymphoma. Blood. 2015; 126(19):2193-201.
32. Kwon D, Kim S, Kim P J, Go H, Nam S J, Paik J H, et al. Clinicopathological analysis of programmed cell death 1 and programmed cell death ligand 1 expression in the tumour microenvironments of diffuse large B cell lymphomas. Histopathology. 2016; 68(7):1079-89.
33. Xing W, Dresser K, Zhang R, Evens A M, Yu H, Woda B A, et al. P D-L1 expression in EBV658 negative diffuse large B-cell lymphoma: clinicopathologic features and prognostic implications. Oncotarget. 2016; 7(37):59976-86.
34. Kwiecinska A, Tsesmetzis N, Ghaderi M, Kis L, Saft L, Rassidakis G Z. CD274 (PD661 L1)/PDCD1 (PD-1) expression in de novo and transformed diffuse large B-cell lymphoma. British journal of haematology. 2016.
35. Park S, Lee J, Ko Y H, Han A, Jun H J, Lee S C, et al. The impact of Epstein-Barr virus status on clinical outcome in diffuse large B-cell lymphoma. Blood. 2007; 110(3):972-8.
36. Roemer M G, Advani R H, Redd R A, Pinkus G S, Natkunam Y, Ligon A H, et al. Classical Hodgkin Lymphoma with Reduced beta2M/MHC Class I Expression Is Associated with Inferior Outcome Independent of 9p24.1 Status. Cancer immunology research. 2016; 4(11):910-6.
37. Green M R, Rodig S, Juszczynski P, Ouyang J, Sinha P, O'Donnell E, et al. Constitutive AP-1 activity and EBV infection induce P D-L1 in Hodgkin lymphomas and posttransplant lymphoproliferative disorders: implications for targeted therapy. Clinical cancer research: an official journal of the American Association for Cancer Research. 2012; 18(6):1611-8.25
38. Fang W, Zhang J, Hong S, Zhan J, Chen 672 N, Qin T, et al. EBV-driven LMP1 and IFN-gamma up-regulate P D-L1 in nasopharyngeal carcinoma: Implications for oncotargeted therapy. Oncotarget. 2014; 5(23):12189-202.
39. Trivedi P, Zhang Q J, Chen F, Minarovits J, Ekman M, Biberfeld P, et al. Parallel existence of Epstein-Barr virus (EBV) positive and negative cells in a sporadic case of Burkitt lymphoma. Oncogene. 1995; 11(3):505-10.
40. Kempkes B, Spitkovsky D, Jansen-Durr P, Ellwart J W, Kremmer E, Delecluse H J, et al. Bcell proliferation and induction of early G1-regulating proteins by Epstein-Barr virus mutants conditional for EBNA2. The EMBO journal. 1995; 14(1):88-96.
41. Anastasiadou E, Vaeth S, Cuomo L, Boccellato F, Vincenti S, Cirone M, et al. Epstein-Barr virus infection leads to partial phenotypic reversion of terminally differentiated malignant B cells. Cancer letters. 2009; 284(2):165-74.
42. Anastasiadou E, Garg N, Bigi R, Yadav S, Campese A F, Lapenta C, et al. Epstein-Barr virus infection induces miR-21 in terminally differentiated malignant B cells. International journal of cancer. 2015; 137(6):1491-7.
43. Veroni C, Marnetto F, Granieri L, Bertolotto A, Ballerini C, Repice A M, et al. Immune and Epstein-Barr virus gene expression in cerebrospinal fluid and peripheral blood mononuclear cells from patients with relapsing-remitting multiple sclerosis. Journal of neuroinflammation. 2015; 12:132.
44. Kempkes B, Pawlita M, Zimber-Strobl U, Eissner G, Laux G, Bornkamm G W. Epstein-Barr virus nuclear antigen 2-estrogen receptor fusion proteins transactivate viral and cellular genes and interact with RBP-J kappa in a conditional fashion. Virology. 1995; 214(2):675-9.
45. Imai S, Nishikawa J, Takada K. Cell-to-cell contact as an efficient mode of Epstein-Barr virus infection of diverse human epithelial cells. Journal of virology. 1998; 72(5):4371-8.
46. Anastasiadou E, Boccellato F, Vincenti S, Rosato P, Bozzoni I, Frati L, et al. Epstein-Barr virus encoded LMP1 downregulates TCL1 oncogene through miR-29b. Oncogene. 2010; 29(9): 1316-28.
47. Anastasiadou E, Boccellato F, Cirone M, Kis L L, Klein E, Frati L, et al. Epigenetic mechanisms do not control viral latency III in primary effusion lymphoma cells infected with a recombinant Epstein-Barr virus. Leukemia. 2005; 19(10):1854-6.
48. Di Napoli A, Al-Jadiri M F, Talerico C, Duranti E, Pilozzi E, Trivedi P, et al. Epstein-Barr virus (EBV) positive classical Hodgkin lymphoma of Iraqi children: an immunophenotypic and molecular characterization of Hodgkin/Reed-Sternberg cells. Pediatric blood & cancer. 2013; 60(12):2068-72.
49. Salzman D W, Nakamura K, Nallur S, Dookwah M T, Metheetrairut C, Slack F J, et al. miR-34 activity is modulated through 5′-end phosphorylation in response to DNA damage. Naturecommunications. 2016; 7:10954.
50. Adams B D, Wali V B, Cheng C J, Inukai S, Booth C J, Agarwal S, et al. miR-34a Silences c-SRC to Attenuate Tumor Growth in Triple-Negative Breast Cancer. Cancer Res. 2016; 76(4):927-39.
51. Alimperti S, Mirabella T, Bajaj V, Polacheck W, Pirone D M, Duffield J, et al. Three dimensional biomimetic vascular model reveals a RhoA, Racl, and N-cadherin balance in mural cell-endothelial cell-regulated barrier function. Proceedings of the National Academy of Sciences of the United States of America. 2017; 114(33):8758-63.
52. Nguyen D H, Stapleton S C, Yang M T, Cha S S, Choi C K, Galie P A, et al. Biomimetic model to reconstitute angiogenic sprouting morphogenesis in vitro. Proc Natl Acad Sci USA. 2013; 110(17):6712-7.
53. Trivedi P, Spinsanti P, Cuomo L, Volpe M, Takada K, Frati L, et al. Differential regulation of Epstein-Barr virus (EBV) latent gene expression in Burkitt lymphoma cells infected with a recombinant EBV strain. Journal of virology. 2001; 75(10):4929-35.
54. Rosato P, Anastasiadou E, Ga 722 rg N, Lenze D, Boccellato F, Vincenti S, et al. Differential regulation of miR-21 and miR-146a by Epstein-Barr virus-encoded EBNA2. Leukemia. 2012; 26(11):2343-52.
55. Kasinski A L, Slack F J. miRNA-34 prevents cancer initiation and progression in a therapeutically resistant K-ras and p53-induced mouse model of lung adenocarcinoma. Cancer research. 2012; 72(21):5576-87.
56. Khan A, Fornes O, Stigliani A, Gheorghe M, Castro-Mondragon J A, van der Lee R, et al. JASPAR 2018: update of the open-access database of transcription factor binding profiles and its web framework. Nucleic acids research. 2017.
57. Thorvaldsdottir H, Robinson J T, Mesirov J P. Integrative Genomics Viewer (IGV): high732 performance genomics data visualization and exploration. Briefings in bioinformatics. 2013; 14(2): 178-92.
58. Ploegh H L. Viral strategies of immune evasion. Science (New York, N.Y.). 1998; 280(5361):248-53.
59. Blenk S, Engelmann J, Weniger M, Schultz J, Dittrich M, Rosenwald A, et al. Germinal center B cell-like (GCB) and activated B cell-like (ABC) type of diffuse large B cell lymphoma (DLBCL): analysis of molecular predictors, signatures, cell cycle state and patient survival. Cancer informatics. 2007; 3:399-420.
60. Bader A G. miR-34—a microRNA replacement therapy is headed to the clinic. Frontiers in genetics. 2012; 3:120.
61. Marques S C, Ranjbar B, Laursen M B, Falgreen S, Bilgrau A E, Bodker J S, et al. High miR-34a expression improves response to doxorubicin in diffuse large B-cell lymphoma. Experimental hematology. 2016; 44(4):238-46.e2.
62. Craig V J, Tzankov A, Flori M, Schmid C A, Bader A G, Muller A. Systemic microRNA-34a delivery induces apoptosis and abrogates growth of diffuse large B-cell lymphoma in vivo. Leukemia. 2012; 26(11):2421-4.
63. Rao D S, O'Connell R M, Chaudhuri A A, Garcia-Flores Y, Geiger T L, Baltimore D. MicroRNA-34a perturbs B lymphocyte development by repressing the forkhead box transcription factor Foxpl. Immunity. 2010; 33(1):48-59.
64. Pyzer A R, Stroopinsky D, Rosenblatt J, Anastasiadou E, Rajabi H, Washington A, et al. MUC1 inhibition leads to decrease in P D-L1 levels via up-regulation of miRNAs. Leukemia. 2017.
65. Wille C K, Li Y, Rui L, Johannsen E C, Kenney S C. Restricted TET2 Expression in Germinal Center Type B Cells Promotes Stringent Epstein-Barr Virus Latency. Journal of virology. 2017; 91(5).
66. Lu F, Wiedmer A, Martin K A, Wickramasinghe P, Kossenkov A V, Lieberman P M. Coordinate Regulation of TET2 and EBNA2 Control DNA Methylation State of Latent Epstein-Barr Virus. Journal of virology. 2017.
67. Li Y, Guessous F, Zhang Y, Dipierro C, Kefas B, Johnson E, et al. MicroRNA-34a inhibits glioblastoma growth by targeting multiple oncogenes. Cancer research. 2009; 69(19):7569-76.
68. Yamamura S, Saini S, Majid S, Hirata H, Ueno K, Chang I, et al. MicroRNA-34a suppresses malignant transformation by targeting c-Myc transcriptional complexes in human renal cell carcinoma. Carcinogenesis. 2012; 33(2):294-300.
69. Casey S C, Tong L, Li Y, Do R, Walz S, Fitzgerald K N, et al. MYC regulates the antitumor immune response through CD47 and P D-L1. Science (New York, N.Y.). 2016; 352(6282):227-31.

What is claimed is:

1. A method for treating an Epstein-Barr virus (EBV)-related cancer in a subject in need thereof, comprising administering an effective amount of one or more of (a) an agent that increases an amount of miR-34a in the subject and (b) an agent that decreases an amount of miR-129 in the subject, wherein:
the subject is undergoing treatment with an immune checkpoint immunotherapy selected from an agent that modulates one or more of programmed cell death protein-1 (PD-1), programmed death-ligand 1 (PD-L1), programmed death-ligand 2 (PD-L2), inducible T-cell costimulator (ICOS), inducible T-cell costimulator ligand (ICOSL), and cytotoxic T-lymphocyte-associated protein 4 (CTLA-4).

2. The method of claim 1, wherein the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), and adult T-cell leukemia (ATL)/lymphoma.

3. The method of claim 1, wherein the agent that increases an amount of miR-34a is selected from one or more of miR-34a and a miR-34a mimetic.

4. The method of any of the above claims, wherein the agent that increases an amount of miR-34a is an inhibitor of Early B-cell factor (EBF1).

5. The method of claim 1, wherein the agent that decreases an amount of miR-129 is selected from one or more of an antisense oligonucleotide, an antagomir, and a construct expressing a miRNA inhibitor.

6. The method of claim 5, wherein the antisense oligonucleotide comprises a sequence that is at least partially complementary to a mature sequence of miR-129.

7. The method of claim 5, wherein one or more nucleotides of the agent are chemically modified.

8. The method of claim 7, wherein the chemical modification is selected from locked nucleic acid (LNA), phosphorothioate, 2′-O-Methyl, 2′-O-Methoxyethyl, 2′-O-alkyl-RNA unit, 2′-OMe-RNA unit, 2′-amino-DNA unit, 2′-fluoro-DNA unit, peptide nucleic acid (PNA) unit, hexitol nucleic acids (HNA) unit, INA unit, and a 2′-O-(2-Methoxyethyl)-RNA (2′ MOE RNA) unit.

9. The method of any of the above claims, wherein the agent that modulates PD-1 is an antibody or antibody format specific for PD-1.

10. The method of claim 9, wherein the antibody or antibody format specific for PD-1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

11. The method of claim 9, wherein the antibody or antibody format specific for PD-1 is selected from Nivolumab, Pembrolizumab, Pidilizumab, BMS-936559, Atezolizumab, or Avelumab.

12. The method of any of the above claims, wherein the agent that modulates PD-L1 is an antibody or antibody format specific for PD-L1.

13. The method of claim 12, wherein the antibody or antibody format specific for PD-L1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

14. The method of claim 12, wherein the antibody or antibody format specific for PD-L1 is selected from Nivolumab, Pembrolizumab, Pidilizumab, BMS-936559, Atezolizumab, Avelumab or Durvalumab.

15. The method of any of the above claims, wherein the agent that modulates PD-L2 is an antibody or antibody format specific for PD-L2.

16. The method of claim 15, wherein the antibody or antibody format specific for PD-L2 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

17. The method of any of the above claims, wherein the agent that modulates ICOS is an antibody or antibody format specific for ICOS.

18. The method of claim 17, wherein the antibody or antibody format specific for ICOS is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

19. The method of claim 17, wherein the antibody or antibody format specific for ICOS comprises JTX-2011.

20. The method of any of the above claims, wherein the agent that modulates ICOSL is an antibody or antibody format specific for ICOSL.

21. The method of claim 20, wherein the antibody or antibody format specific for ICOSL is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

22. The method of any of the above claims, wherein the agent that modulates CTLA-4 is an antibody or antibody format specific for CTLA-4.

23. The method of claim 22, wherein the antibody or antibody format specific for CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

24. The method of claim 22, wherein the antibody or antibody format specific for CTLA-4 is selected from tremelimumab or ipilimumab.

25. The method of any of the above claims, wherein administration is by intratumoral, intradermal, subcutaneous, intramuscular, intraperitoneal or intravenous injection, or direct injection into cancer tissue.

26. A method for treating an EBV-related cancer in a subject in need thereof, comprising administering (i) an effective amount of one or more of (a) an agent that increases an amount of miR-34a in the subject and (b) an agent that decreases an amount of miR-129 in the subject, and (ii) an effective amount of an immune checkpoint immunotherapy selected from an agent that modulates one or more of PD-1, PD-L1, PD-L2, ICOS, ICOSL, and CTLA-4.

27. The method of claim 26, wherein the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), and adult T-cell leukemia (ATL)/lymphoma.

28. The method of claim 26 or 27, wherein the agent that increases an amount of miR-34a is selected from one or more of miR-34a and a miR-34a mimetic.

29. The method of any one of claims 26-28, wherein the agent that increases an amount of miR-34a is an inhibitor of Early B-cell factor (EBF1).

30. The method of claim 26 or 27, wherein the agent that decreases an amount of miR-129 is selected from one or more of an antisense oligonucleotide, an antagomir and a construct expressing a miRNA inhibitor.

31. The method of claim 30, wherein the antisense oligonucleotide comprises a sequence that is at least partially complementary to a mature sequence of miR-129.

32. The method of claim 31, wherein one or more nucleotides of the agent are chemically modified.

33. The method of claim 32, wherein the chemical modification is selected from locked nucleic acid (LNA), phosphorothioate, 2′-O-Methyl, 2′-O-Methoxyethyl, 2′-O-alkyl-RNA unit, 2′-OMe-RNA unit, 2′-amino-DNA unit, 2′-fluoro-DNA unit, peptide nucleic acid (PNA) unit, hexitol nucleic acids (HNA) unit, INA unit, and a 2′-O-(2-Methoxyethyl)-RNA (2′ MOE RNA) unit.

34. The method of any one of claims 26-33, wherein the agent that modulates PD-1 is an antibody or antibody format specific for PD-1.

35. The method of claim 34, wherein the antibody or antibody format specific for PD-1 is selected from one or more of wherein the antibody or antibody format specific for PD-1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

36. The method of claim 34, wherein the antibody or antibody format specific for PD-1 is selected from Nivolumab, Pembrolizumab, Pidilizumab, BMS-936559, Atezolizumab, or Avelumab.

37. The method of any one of claims 26-36, wherein the agent that modulates PD-L1 is an antibody or antibody format specific for PD-L1.

38. The method of claim 37, wherein the antibody or antibody format specific for PD-L1 is selected from one or more of is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

39. The method of claim 37, wherein the antibody or antibody format specific for PD-L1 is selected from Nivolumab, Pembrolizumab, Pidilizumab, BMS-936559, Atezolizumab, Avelumab or Durvalumab.

40. The method of any one of claims 26-39, wherein the agent that modulates PD-L2 is an antibody or antibody format specific for PD-L2.

41. The method of claim 40, wherein the antibody or antibody format specific for PD-L2 is selected from one or more of is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

42. The method of any one of claims 26-41, wherein the agent that modulates ICOS is an antibody or antibody format specific for ICOS.

43. The method of claim 42, wherein the antibody or antibody format specific for ICOS is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody

44. The method of claim 42, wherein the antibody or antibody format specific for ICOS comprises JTX-2011.

45. The method of any one of claims 26-44, wherein the agent that modulates ICOSL is an antibody or antibody format specific for ICOSL.

46. The method of claim 45, wherein the antibody or antibody format specific for ICOSL is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, or fusion protein comprising the antigen-binding portion of an antibody.

47. The method of any one of claims 26-46, wherein the agent that modulates CTLA-4 is an antibody or antibody format specific for CTLA-4.

48. The method of claim 47, wherein the antibody or antibody format specific for CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

49. The method of claim 47, wherein the antibody or antibody format specific for CTLA-4 is selected from tremelimumab or Ipilimumab.

50. The method of any one of claims 26-49, wherein the administration is sequential.

51. The method of any one of claims 26-50, wherein the effective amount of one or more of (a) agent that increases an amount of miR-34a and (b) agent that decreases an amount of miR-129 is administered before the effective amount of the immune checkpoint immunotherapy.

52. The method of any one of claim 26-50, wherein the effective amount of the immune checkpoint immunotherapy is administered before the effective amount of one or more of (a) agent that increases an amount of miR-34a and (b) agent that decreases an amount of miR-129.

53. The method of any one of claim 26-49, wherein the administration is simultaneous.

54. The method of any one of claim 26-53, wherein administration is intratumoral, intradermal, subcutaneous, intramuscular, intraperitoneal or intravenous injection, or by direct injection into cancer tissue.

55. The method of any one of claims 26-54, wherein the effective amount of the immune checkpoint immunotherapy and effective amount of one or more of (a) agent that increases an amount of miR-34a and (b) agent that decreases an amount of miR-129 are co-formulated on a nanoparticle, nanostructured polymer or a biopolymer.

56. A method potentiating an immune checkpoint immunotherapy of an EBV-related cancer in a subject in need thereof, comprising administering an agent that increases an amount of miR-34a in the subject, wherein:
the immune checkpoint immunotherapy is an agent that modulates one or more of PD-1, PD-L1, and PD-L2 and the subject is predicted to be poorly responsive or non-responsive to the immune checkpoint immunotherapy or has presented as poorly responsive or non-responsive to the immune checkpoint immunotherapy.

57. The method of claim 56, wherein the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), and adult T-cell leukemia (ATL)/lymphoma).

58. The method of claim 56 or 57, wherein the agent that increases an amount of miR-34a is selected from one or more of miR-34a and a miR-34a mimetic.

59. The method of any one of claims 56-58, wherein the agent that increases an amount of miR-34a is an inhibitor of Early B-cell factor (EBF1).

60. The method of claim 56 or 57, wherein the agent that decreases an amount of miR-129 is selected from one or more of an antisense oligonucleotide, an antagomir, and a construct expressing a miRNA inhibitor.

61. The method of claim 60, wherein the antisense oligonucleotide comprises a sequence that is at least partially complementary to a mature sequence of miR-129.

62. The method of claim 60, wherein one or more nucleotides of the agent are chemically modified.

63. The method of claim 62, wherein the chemical modification is selected from locked nucleic acid (LNA), phosphorothioate, 2′-O-Methyl, 2′-O-Methoxyethyl, 2′-O-alkyl-RNA unit, 2′-OMe-RNA unit, 2′-amino-DNA unit, 2′-fluoro-DNA unit, peptide nucleic acid (PNA) unit, hexitol nucleic acids (HNA) unit, INA unit, and a 2′-O-(2-Methoxyethyl)-RNA (2′ MOE RNA) unit.

64. The method of any one of claims 56-63, wherein the agent that modulates PD-1 is an antibody or antibody format specific for PD-1.

65. The method of claim 64, wherein the antibody or antibody format specific for PD-1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

66. The method of claim 64, wherein the antibody or antibody format specific for PD-1 is selected from Nivolumab, Pembrolizumab, Pidilizumab, BMS-936559, Atezolizumab, or Avelumab.

67. The method of any one of claims 56-66, wherein the agent that modulates PD-L1 is an antibody or antibody format specific for PD-L1.

68. The method of claim 67, wherein the antibody or antibody format specific for PD-L1 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

69. The method of claim 67, wherein the antibody or antibody format specific for PD-L1 is selected from Nivolumab, Pembrolizumab, Pidilizumab, BMS-936559, Atezolizumab, Avelumab or Durvalumab.

70. The method of any one of claims 56-69, wherein the agent that modulates PD-L2 is an antibody or antibody format specific for PD-L2.

71. The method of claim 70, wherein the antibody or antibody format specific for PD-L2 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

72. A method potentiating immune checkpoint immunotherapy of an EBV-related cancer in a subject in need thereof, comprising administering an agent that decreases an amount of miR-129 in the subject, wherein:
the immune checkpoint immunotherapy is an agent that modulates one or more of ICOS, ICOSL, and CTLA-4 and
the subject is predicted to be poorly responsive or non-responsive to the immune checkpoint immunotherapy or has presented as poorly responsive or non-responsive to the immune checkpoint immunotherapy.

73. The method of claim 72, wherein the EBV-related cancer is selected from one or more of Non-Hodgkin lymphoma (NHL), B-cell Lymphoma (BL), Burkitt lymphoma, Hodgkin lymphoma (HL), nasopharyngeal carcinoma, gastric carcinoma, human T-lymphotropic virus 1 (HTLV-1), and adult T-cell leukemia (ATL)/lymphoma.

74. The method of claim 72 or 73, wherein the agent that increases an amount of miR-34a is selected from one or more of miR-34a and a miR-34a mimetic.

75. The method of any one of claims 72-74, wherein the agent that increases an amount of miR-34a is an inhibitor of Early B-cell factor (EBF1).

76. The method of claim 72 or 73, wherein the agent that decreases an amount of miR-129 is selected from one or more of an antisense oligonucleotide, an antagomir, and a construct expressing a miRNA inhibitor.

77. The method of claim 76, wherein the antisense oligonucleotide comprises a sequence that is at least partially complementary to a mature sequence of miR-129.

78. The method of claim 76, wherein one or more nucleotides of the agent are chemically modified.

79. The method of claim 78, wherein the chemical modification is selected from locked nucleic acid (LNA), phosphorothioate, 2′-O-Methyl, 2′-O-Methoxyethyl, 2′-O-alkyl-RNA unit, 2′-OMe-RNA unit, 2′-amino-DNA unit, 2′-fluoro-DNA unit, peptide nucleic acid (PNA) unit, hexitol nucleic acids (HNA) unit, INA unit, and a 2′-O-(2-Methoxyethyl)-RNA (2′ MOE RNA) unit.

80. The method of any one of claims 72-79, wherein the agent that modulates ICOS is an antibody or antibody format specific for ICOS.

81. The method of claim 80, wherein the antibody or antibody format specific for ICOS is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

82. The method of claim 81, wherein the antibody or antibody format specific for ICOS comprises JTX-2011.

83. The method of any one of claims 72-82, wherein the agent that modulates ICOSL is an antibody or antibody format specific for ICOSL.

84. The method of claim 83, wherein the antibody or antibody format specific for ICOSL is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

85. The method of any one of claims 72-84, wherein the agent that modulates CTLA-4 is an antibody or antibody format specific for CTLA-4.

86. The method of claim 85, wherein the antibody or antibody format specific for CTLA-4 is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

87. The method of claim 85, wherein the antibody or antibody format specific for CTLA-4 is selected from tremelimumab or Ipilimumab.

88. The method of any of the above claims, wherein the method reduces and/or mitigates one or more side effects of the immune checkpoint immunotherapy.

89. The method of claim 88, wherein the side effect is selected from decreased appetite, rashes, fatigue, pneumonia, pleural effusion, pneumonitis, pyrexia, nausea, dyspnea, cough, constipation, diarrhea, immune-mediated pneumonitis, colitis, hepatitis, endocrinopathies, hypophysitis, iridocyclitis, and nephritis.

90. The method of any of the above claims, wherein the method reduces the dose of immune checkpoint immunotherapy.

91. The method of any of the above claims, wherein the method reduces number of administrations of the immune checkpoint immunotherapy.

92. The method of any of the above claims, wherein the method increases a therapeutic window of the immune checkpoint immunotherapy.

93. The method of any of the above claims, wherein the method elicits a potent immune response in less-immunogenic tumors.

94. The method of any of the above claims, wherein the method converts a tumor with reduced inflammation (“cold tumor”) to a responsive, inflamed tumor (“hot tumor”).

95. The method of any of the above claims, wherein the method makes the cancer responsive or more responsive to a combination therapy of the immune checkpoint immunotherapy and one or more chemotherapeutic agents and/or radiotherapy.

96. The method of claim 95, wherein the chemotherapeutic agent is selected from one or more of daunorubicin, doxorubicin, epirubicin, idarubicin, adriamycin, vincristine, carmustine, cisplatin, 5-fluorouracil, tamoxifen, prodasone, sandostatine, mitomycin C, foscarnet, paclitaxel, docetaxel, gemcitabine, fludarabine, carboplatin, leucovorin, tamoxifen, goserelin, ketoconazole, leuprolide flutamide, vinblastine, vindesine, vinorelbine, camptothecin, topotecan, irinotecan hydrochloride, etoposide, mitoxantrone, teniposide, amsacrine, merbarone, piroxantrone hydrochloride, methotrexate, 6-mercaptopurine, 6-thioguanine, cytarabine (Ara-C), trimetrexate, acivicin, alanosine, pyrazofurin, pentostatin, 5-azacitidine, 5-azacitidine, 5-Aza-5-Aza-2′-deoxycytidine, adenosine arabinoside (Ara-A), cladribine, ftorafur, UFT (combination of uracil and florafur), 5-fluoro-2′-deoxyuridine, 5-fluorouridine, 5′-deoxy-5-fluorouridine, hydroxyurea, dihydrolenchiorambucil, tiazofurin, oxaliplatin, melphalan, thiotepa, busulfan, chlorambucil, plicamycin, dacarbazine, ifosfamide phosphate, cyclophosphamide, pipobroman, 4-ipomeanol, dihydrolenperone, spiromustine, geldenamycin, cytochalasins, depsipeptide, 4′-cyano-3-(4-(e.g., ZOLADEX) and 4′-cyano-3-(4-fluorophenylsulphonyl)-2-hydroxy-3-methyl-3′-(trifluorometh-yl)propionanilide.

97. The method of any of the above claims, wherein the subject is predicted to be poorly responsive or non-responsive to the immune checkpoint immunotherapy based on expression of one or more of PD-1, PD-L1, PD-L2, ICOS, ICOSL, and CTLA-4 in a tumor specimen.

98. The method of claim 97, wherein the subject is predicted to be poorly responsive or non-responsive to an agent that modulates one or more of PD-1, PD-L1, and PD-L2 based on low on expression of PD-1, PD-L1, and PD-L2 in a tumor specimen.

99. The method of any of claim 97 or 98, wherein the subject is predicted to be poorly responsive or non-responsive to an agent that modulates one or more of PD-1, PD-L1, and PD-L2 tumor proportion score (TPS) of less than about 49% for PD-L1 staining.

100. A method for treating an EBV-related cancer in a subject in need thereof, comprising administering (i) an effective amount of one or more of (a) an agent that increases an amount of miR-34a in the subject and (b) an agent that decreases an amount of miR-129 in the subject, and (ii) an effective amount of an immune checkpoint immunotherapy selected from an agent that modulates one or more of PD-1, PD-L1, PD-L2, ICOS, ICOSL, and CTLA-4.

101. A method for evaluating an EBV-related cancer subject's likelihood of response to an immune checkpoint immunotherapy, comprising evaluating a level of one or more of miR-34a and miR-129 in a biological sample from the subject, wherein a low level of miR-34a and/or high level of miR-129 is indicative of a cancer that is suitable for immune checkpoint immunotherapy.

102. A method for treating an EBV-related cancer, comprising:
(a) evaluating a subject's likelihood of response to an immune checkpoint immunotherapy, comprising evaluating a level of one or more of miR-34a and miR-129 in a biological sample from the subject, wherein a low level of miR-34a and/or high level of miR-129 is indicative of a cancer that is suitable for immune checkpoint immunotherapy and
(b) administering an immune checkpoint immunotherapy selected from an agent that modulates one or more of PD-1, PD-L1, and PD-L2 based on low on expression of PD-1, PD-L1, and PD-L2 to the subject having a low level of miR-34a and/or high level of miR-129.

103. The method of claim 102, wherein a high level of miR-34a indicates a cancer that is evading an anti-cancer immune response through a negative immune signal mediated by one or more of PD-1, PD-L1, and PD-L2.

104. The method of claim 102, wherein a low level of miR-129 indicates a cancer that is prevented from delivering a positive anti-tumor signal mediated by one or more of ICOS and ICOSL.

105. The method of claim 102, wherein a low level of miR-34a indicates a high likelihood of response to an immune checkpoint immunotherapy selected from an agent that modulates one or more of PD-1, PD-L1, and PD-L2.
